(19) United States
(12) Patent Application Publication
(10) Pub. No.: US 2026/0260941 A1
(43) Pub. Date: Sep. 3, 2026

(54) ADDITIVES FOR ELECTROLYTES IN Li-ION BATTERIES

(71) Applicants: HYDRO-QUÉBEC, Montréal (CA); MURATA MANUFACTURING CO., LTD., Nagaokakyo-shi (JP)

(72) Inventors: Charlotte Mallet, Montréal (CA); Sylviane Rochon, Saint-Adélphe (CA); Karim Zaghib, Longueuil (CA); David Lepage, Sainte-Julie (CA); Nicolas Dumaresq, Valleyfield (CA)

(73) Assignees: HYDRO-QUÉBEC, Montréal (CA); MURATA MANUFACTURING CO., LTD., Nagaokakyo-shi (JP)

(21) Appl. No.: 19/655,067

(22) Filed: Apr. 22, 2026

Related U.S. Application Data
(63) Continuation-in-part of application No. 18/893,679, filed on Sep. 23, 2024, now Pat. No. 12,614,756.
Application No. 18/893,679 is a continuation of application No. 17/281,120, filed on Oct. 3, 2019, now Pat. No. 12,100,807.
(60) Provisional application No. 62/741,275, filed on Oct. 4, 2018.

(51) Int. Cl. H01M 10/0567; C07C 255/50; C07C 255/51; C07C 255/55; C07D 213/57; H01M 10/0525; H01M 10/42

(52) U.S. Cl. CPC H01M 10/0567; C07C 255/50; C07C 255/51; C07C 255/55; C07D 213/57; H01M 10/0525; H01M 10/4235

(57) ABSTRACT

Methods of improving the performance and safety of a Li-ion battery are provided herein. One such method comprises using a nitrile-based small organic compound of general formula A0 in association with the electrolyte of the battery. Also provided herein is an electrolyte including the nitrile-based small organic compound and a battery including the electrolyte.

### CROSS-REFERENCE TO RELATED APPLICATIONS

This application is a Continuation-In-Part (CIP) Application of U.S. patent application Ser. No. 18/893,679, filed Sep. 23, 2024, titled “ADDITIVES FOR ELECTROLYTES IN Li-ION BATTERIES,” now U.S. Pat. No. 12,614,756, issued Apr. 28, 2026, which is a continuation of U.S. patent application Ser. No. 17/281,120, filed Mar. 29, 2021, titled “ADDITIVES FOR ELECTROLYTES IN Li-ION BATTERIES,” now U.S. Pat. No. 12,100,807, issued Sep. 24, 2024, which is a U.S. national stage application of International Application No. PCT/CA2019/051415, filed Oct. 3, 2019, titled “ADDITIVES FOR ELECTROLYTES IN Li-ION BATTERIES,” which claims priority to and the benefit of U.S. Provisional Patent Application No. 62/741,275, filed Oct. 4, 2018, the contents of which are hereby incorporated by reference in their entireties.

### FIELD OF THE INVENTION

The present invention relates generally to additives for Li-ion batteries. More specifically, the present invention relates to nitrile-based additives for use in association with the electrolyte in Li-ion batteries.

### BACKGROUND OF THE INVENTION

Li-ion batteries are widely used as energy source, and the demand is increasing. Typically, such battery comprises a negative electrode or anode, a positive electrode or cathode, and an electrolyte provided between the two spaced-apart electrodes. The electrolyte may comprise organic molecules or polymers and generally also comprises a lithium salt such as LiPF₆, LiTFSI or LiFSI. Moreover, the electrolyte may comprise linear carbonates such as dimethyl carbonate (DMC), diethyl carbonate (DEC), ethylmethyl carbonate (EMC) or cyclic carbonates such as ethylene carbonate (EC), propylene carbonate (PC) and butylene carbonate (BC).

Various studies related to the nature and composition of electrolytes and aimed at improving the performance and safety of Li-ion batteries, are reported in the art. For example, the use of additives comprising one or more nitrile groups is reported [1-3]. Indeed, it is known in the art that organic compounds comprising nitrile groups present good electrochemical properties and stability at high voltage and temperature.

Despite solid performance levels, there is a strong demand for enhancing the cyclability and energy density of lithium batteries. This is particularly important for electric vehicles, which require longer ranges and less frequent battery replacements. Various strategies can be employed to address these challenges, such as utilizing new materials, optimizing pack cell design, and incorporating additives. The use of additives, in particular, allows for improvements while maintaining existing processes and assembly lines. Additives can have many uses e.g. safety such as flame-retarding [4], high-voltage [5], redox-shuffle [6], gas reduction [7], enhancing or stabilizing solid-electrolyte-interphase (SEI) [8] or cathode-electrolyte-interphase (CEI) [9]. The latter is important for stabilizing the surface of Ni-rich such LiNi_{0.8}Mn_{0.1}Co_{0.1}O₂ (NMC811) cathode materials [10]. Indeed, applying high voltage at the cathode promotes the formation of the cathode-electrolyte interphase (CEI), primarily due to electrolyte decomposition at the cathode surface. This phenomenon is particularly critical for high-nickel cathode materials, such as NMC811, where gas evolution has been observed even at relatively moderate potentials, as low as 4.4 V [11]. A well-formed CEI is therefore expected to mitigate further parasitic reactions involving the electrolyte as well as preventing the dissolution of the metal. The formation of a robust and homogeneous CEI is critical for achieving improved long-term cycling performance [12]. As example, Xia et al. have increased the cyclability from 200 to 800 cycles by adding 2% of prop-1-ene, 1,3-sultone (PES)+1% 1,3,2-dioxathiolan-2,2-oxide (DTD) in a graphite/LiNi_{0.4}Mn_{0.4}Co_{0.2}O₂ system [13]. Another example is Qiu et al. show an increase of 17.6% after 200 cycles by adding 0.5% of adiponitrile (ADN) with graphite/NMC811 system [14].

The development of new molecules as additives remains difficult because the vast array of options, which complicates the research and development process [15, 16]. There is an almost infinite number of possibilities among additives, including fluorinated [13], boronated [17], organosilane-based [18], or other carbon-based molecules. However, some additives such as vinylene carbonate (VC) and nitrile-based compounds are already widely recognized for their effectiveness in battery technology. Vinylene carbonate (VC) is indeed a well-known additive that significantly enhances the performance of graphite electrodes, especially at elevated temperatures. It helps in forming a stable solid electrolyte interphase (SEI), which is crucial for maintaining the integrity and efficiency of the electrode during high-temperature operation [19]. For the nitrile-based additives, several compounds have already been utilized, such as adiponitrile [14], 3,3-(Ethylenedioxy) dipropiononitrile [20], 3-, 4-(Trifluoromethyl)-benzonitrile and 1,3,6-Hexanetricarbonitrile [22]. In these papers, it can be observed that nitrile additives are primarily recognized for their ability to aid in the formation of a stable cathode electrolyte interphase (CEI). Therefore, combining VC with a nitrile compound can provide robust SEI and CEI.

There is still a need for methods of improving the performance and safety of Li-ion batteries. In particular, there is a need for nitrile-based organic compounds for use as additives in electrolytes.

### SUMMARY OF THE INVENTION

The inventors have designed and prepared an additive for use in association with the electrolyte in a Li-ion battery. The additive of the invention is an organic compound as described herein and which comprises at least one nitrile group. The organic compound is compatible with the electrolyte as well as other components of the battery. Also, an aspect of the invention is the use of 3,4-dimethoxybenzonitrile as an additive in the electrolyte. A synergistic effect between the nitrile group and the methoxy groups is noted, which enhances the overall performance of the battery.

The invention thus provides the following in accordance with aspects thereof:
(1) A method of improving the performance and safety of a Li-ion battery, comprising using a nitrile-based organic compound in association with the electrolyte of the battery, wherein the compound has a general formula I outlined below

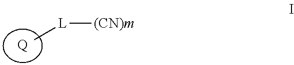

wherein:
   Q is a 5 to 12-member ring or bicycle ring, optionally the ring comprises one or more heteroatom which are the same or different and selected from the group consisting of N, O and S; preferably Q is a 5-10-, or a 5-, or a 6-member ring or bicycle ring;
   L is present or absent and is a linker comprising one or more of alkyl, alkene and alkyne groups; and
   m in an integer from 1 to 10, or 1 to 6, or 1 to 5, or 1 to 4, or 1 to 3.
(2) A method of improving the performance and safety of a Li-ion battery, comprising using a nitrile-based organic compound in association with the electrolyte of the battery, wherein the compound has a general formula II outlined below

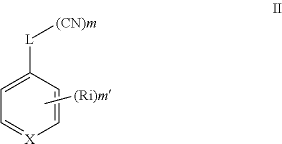

wherein:
   X is C or N;
   L is present or absent and is a linker comprising one or more of alkyl, alkene and
   alkyne groups;
   Ri each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro, and cyano; more preferably selected from the group consisting of H, halogen, nitro and cyano;
   m is an integer from 1 to 5, or 1 to 4, or 1 to 3; and
   m′ is an integer from 0 to 5, or 0 to 4, or 0 to 3, or 1 to 5, or 1 to 4, or 1 to 3.
(3) A method of improving the performance and safety of a Li-ion battery, comprising using a nitrile-based organic compound in association with the electrolyte of the battery, wherein the compound has a general formula III outlined below

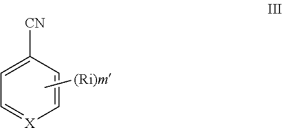

wherein:
   X is C or N;
   Ri are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably Ri are each independently selected from the group consisting of H, alkyloxy, halogen, halogeno alkyl, nitro, and cyano; more preferably selected from the group consisting of H, halogen, nitro and cyano; and
   m′ is an integer from 0 to 5, or 0 to 4, or 0 to 3, or 1 to 5, or 1 to 4, or 1 to 3.
(4) A method of improving the performance and safety of a Li-ion battery, comprising using a nitrile-based organic compound in association with the electrolyte of the battery, wherein the compound has a general formula IV outlined below

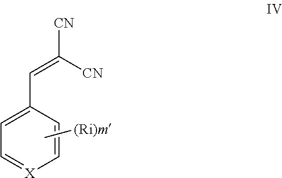

wherein:
   X is C or N;
   Ri are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro and cyano; more preferably selected from the group consisting of H, halogen, nitro and cyano; and
   m′ is an integer from 0 to 5, or 0 to 4, or 0 to 3, or 1 to 5, or 1 to 4, or 1 to 3.
(5) A method of improving the performance and safety of a Li-ion battery, comprising using a nitrile-based organic compound in association with the electrolyte of the battery, wherein the compound has a general formula A outlined below

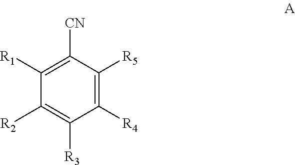

wherein: R₁ to R₅ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably R₁ to R₅ are each independently selected from the group consisting of H, alkyloxy, halogen, halogeno alkyl, nitro and cyano; more preferably selected from the group consisting of H, halogen, nitro and cyano.
(6) A method of improving the performance and safety of a Li-ion battery, comprising using a nitrile-based organic compound in association with the electrolyte of the battery, wherein the compound has a general formula B outlined below

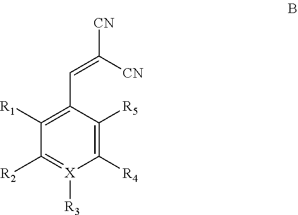

wherein:
   X is C and R₃ is H; or X is N; and
   R₁ to R₅ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably R₁ to R₅ are each independently selected from the group consisting of H, alkyloxy, halogen, halogeno alkyl, nitro and cyano; more preferably selected from the group consisting of H, halogen, nitro and cyano.
(7) A method of improving the performance and safety of a Li-ion battery, comprising using a nitrile-based organic compound in association with the electrolyte of the battery, wherein the compound is A1, A2, A3 or A4 outlined below

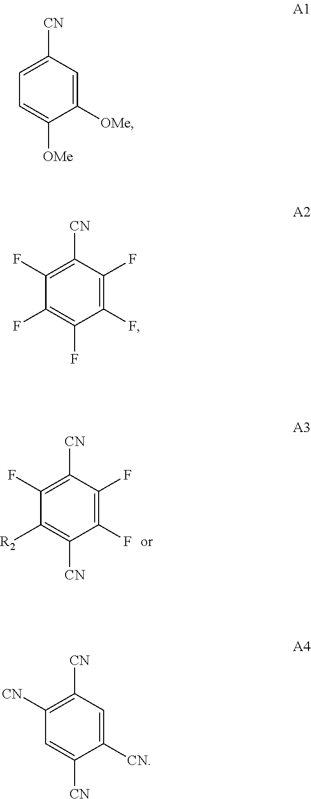

(8) A method of improving the performance and safety of a Li-ion battery, comprising using a nitrile-based organic compound in association with the electrolyte of the battery, wherein the compound is B1, B2, B3, B4, B5, B6, B7 or B8 outlined below

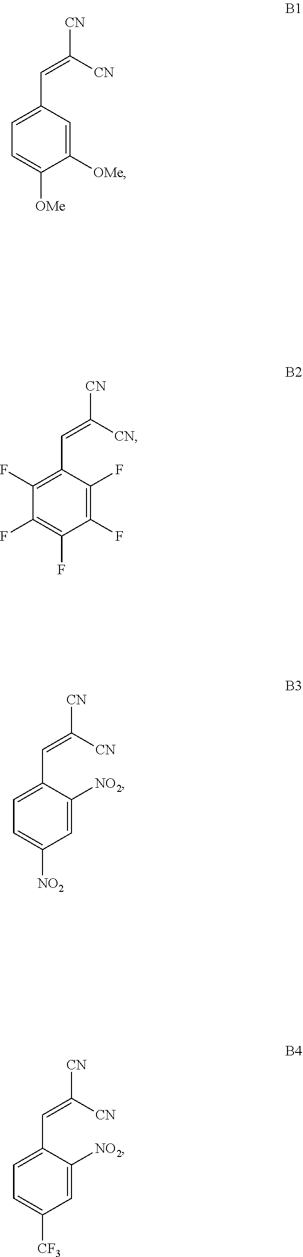 [SMILES: COc1ccc(C=C(C#N)C#N)cc1OC ; N#CC(C#N)=Cc1c(F)c(F)c(F)c(F)c1F ; N#CC(C#N)=Cc1ccc([N+](=O)[O-])cc1[N+](=O)[O-] ; N#CC(C#N)=Cc1ccc(C(F)(F)F)cc1[N+](=O)[O-]]

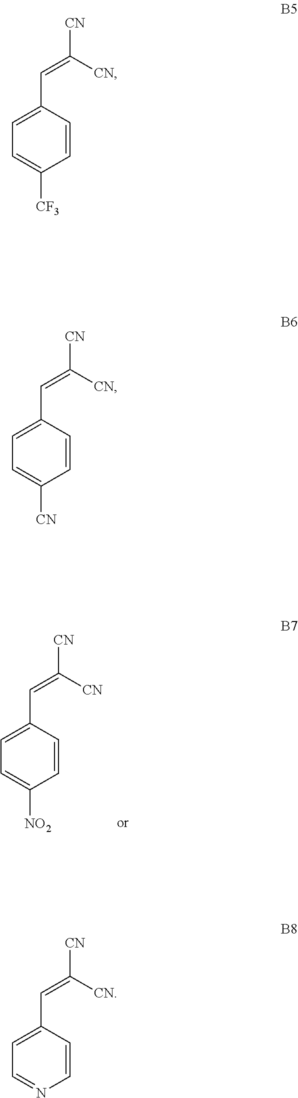 [SMILES: N#CC(C#N)=Cc1ccc(C(F)(F)F)cc1 ; N#CC(C#N)=Cc1ccc(C#N)cc1 ; N#CC(C#N)=Cc1ccc([N+](=O)[O-])cc1 ; N#CC(C#N)=Cc1ccncc1]

(9) A method of improving the performance and safety of a Li-ion battery, comprising using a nitrile-based organic compound in association with the electrolyte of the battery, wherein the compound has a general formula V outlined below

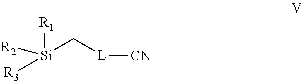

wherein:
   L is present or absent and is a linker comprising one of more of alkyl, alkene and alkyne groups; and
   R₁ to R₃ are each independently alkyl groups; preferably C₁ to C₆ or C₁ to C₃ alkyl groups; more preferably at least one of R₁ to R₃ is CH₃, or each of R₁ to R₃ is CH₃.
(10) A method of improving the performance and safety of a Li-ion battery, comprising using a nitrile-based organic compound in association with the electrolyte of the battery, wherein the compound has a general formula VI outlined below

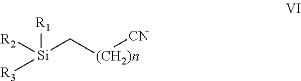

wherein:
   n is an integer from 0 to 6, or 0 to 5, or 0 to 4, or 0 to 3, or 0 to 2; preferably n is an integer from 0 to 3; more preferably n is 0 or 1; and
   R₁ to R₃ are each independently alkyl groups; preferably C₁ to C₆ or C₁ to C₃ alkyl groups; more preferably at least one of R₁ to R₃ is CH₃, or each of R₁ to R₃ is CH₃.
(11) A method of improving the performance and safety of a Li-ion battery, comprising using a nitrile-based organic compound in association with the electrolyte of the battery, wherein the compound has a general formula C outlined below

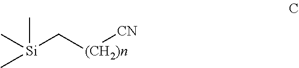

wherein n is an integer from 0 to 6, or 0 to 5, or 0 to 4, or 0 to 3, or 0 to 2; preferably n is an integer from 0 to 3; more preferably n is 0 or 1.
(12) A method of improving the performance and safety of a Li-ion battery, comprising using a nitrile-based organic compound in association with the electrolyte of the battery, wherein the compound is C1 or C2 outlined below

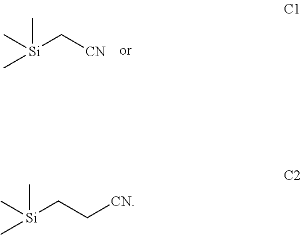

(13) A method of improving the performance and safety of a Li-ion battery, comprising using a nitrile-based organic compound in association with the electrolyte of the battery, wherein the compound has a general formula IX outlined below

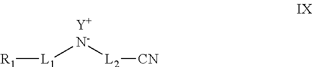

wherein:
   R₁ is CN or CH₃;
   L₁ and L₂ are each independently present or absent and are each independently a linker comprising alkyl, alkene and/or alkyne groups; and
   Y is Na, K or Li; preferably Y is Na.
(14) A method of improving the performance and safety of a Li-ion battery, comprising using a nitrile-based organic compound in association with the electrolyte of the battery, wherein the compound has a general formula X outlined below

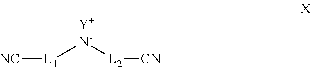

wherein:
   L₁ and L₂ are each independently present or absent and are each independently a linker comprising one or more of alkyl, alkene and alkyne groups; and
   Y is Na, K or Li; preferably Y is Na.
(15) A method of improving the performance and safety of a Li-ion battery, comprising using a nitrile-based organic compound in association with the electrolyte of the battery, wherein the compound has a general formula XI outlined below

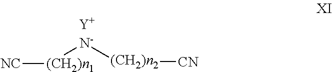

wherein:
   n1 and n2 are each independently an integer from 0 to 10, or 0 to 6, or 0 to 3; preferably at least one of n1 and n2 is 0, or both n1 and n2 are 0; and
   Y is Na, K or Li; preferably Y is Na.
(16) A method of improving the performance and safety of a Li-ion battery, comprising using a nitrile-based organic compound in association with the electrolyte of the battery, wherein the compound has a general formula D outlined below

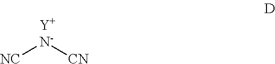

wherein Y is Na, K or Li; preferably Y is Na.
(17) A method of improving the performance and safety of a Li-ion battery, comprising using a nitrile-based organic compound in association with the electrolyte of the battery, wherein the compound has a general formula D1 outlined below

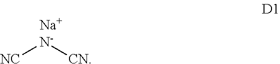

(18) A compound having a general formula VII outlined below

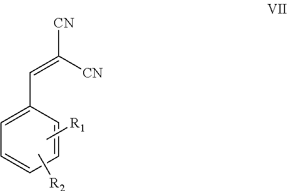

wherein R₁ and R₂ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro and cyano; more preferably selected from the group consisting of H, halogen, nitro and cyano.
(19) A compound having a general formula VIII outlined below

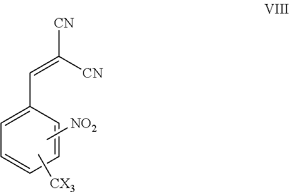

wherein X is a halogen atom; preferably X is F.
(20) A compound of formula B4 outlined below

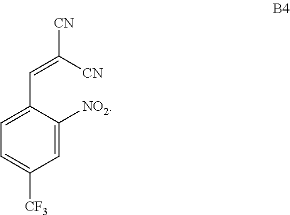 [SMILES: N#CC(C#N)=Cc1ccc(C(F)(F)F)cc1[N+](=O)[O-]]

(21) A method of improving the performance and safety of a Li-ion battery, comprising using a nitrile-based organic compound in association with the electrolyte of the battery, wherein the compound as defined in any one of (18) to (20) above.
(22) The method according to any one of (1) to (17) and (21) above, wherein the nitrile-based organic compound is added to the electrolyte; optionally an amount of the additive (nitrile-based organic compound) is between about 0.01 to about 5.0% wt, or about 0.01 to about 3.0% wt, or about 0.01 to about 1.0% wt, or about 0.05 to about 1.0% wt, or about 0.1 to about 1.0% wt, about 0.1 to about 0.8% wt, or about 0.1 to about 0.5% wt, or about 0.1 to about 0.3% wt, is 0.1% wt, or is 0.5% wt.
(23) An electrolyte comprising a compound which is selected from the group consisting of: I, II, III, IV, A, B, A1, A2, A3, A4, B1, B2, B3, B4, B5, B6, B7, B8, V, VI, C, C1, C2, IX, X, XI, D, and D1 as defined in any one of the methods of (1) to (17) above.
(24) An electrolyte comprising the compound as defined in any one of (18) to (20) above.
(25) A battery comprising the electrolyte as defined in (23) or (24) above.
(26) An additive for an electrolyte for use in a Li-ion battery, comprising a compound which is selected from the group consisting of: I, II, III, IV, A, B, A1, A2, A3, A4, B1, B2, B3, B4, B5, B6, B7, B8, V, VI, C, C1, C2, IX, X, XI, D, and D1 as defined in any one of the methods of (1) to (17) above.
(27) An additive for an electrolyte for use in a Li-ion battery, comprising a compound as defined in any one of (18) to (20) above.
(28) The method, electrolyte, battery or additive according to any one of (1) to (27) above, wherein the Li-ion battery is a battery wherein the cathode comprises a lithium-containing material;
(29) The method, electrolyte, battery or additive according to any one of (1) to (27) above, wherein the Li-ion battery is a battery wherein the cathode comprises lithium cobalt oxide (LCO), lithium manganese oxide (LMO), lithium nickel oxide (LNO) and the like, including olivine's, lithium oxides, nickel manganese cobalt oxide (NMC).
(30) The method, electrolyte, battery or additive according to (28) or (30) above, wherein the performance (capacity, reversibility) of the battery is improved.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1: Cycling data of LMFP-LTO battery (PC/EMC/DMC (4/3/3)+1M LiPF₆+0.1 wt % additive according to the invention (a compounds of Serie A)) versus Reference after 300 cycles at 45° C.

FIG. 2: Static capacity (0.05C) of LMFP-LTO battery (PC/EMC/DMC (4/3/3)+1M LiPF₆+0.1 wt % additive according to the invention (a compound of Serie A)) versus Reference at 45° C.

FIG. 3: Nyquist plots of LMFP-LTO battery (PC/EMC/DMC (4/3/3)+1M LiPF₆+0.1 wt % additive according to the invention (a compound of Serie A)) versus Reference, at 0 and 100 cycles.

FIG. 4: Cycling data of LMFP-LTO battery (PC/EMC/DMC (4/3/3)+1M LiPF₆+0.5 wt % additive according to the invention (a compounds of Serie B)) versus Reference after 300 cycles at 45° C.

FIG. 5: Static capacity (0.05C) of LMFP-LTO battery (PC/EMC/DMC (4/3/3)+1M LiPF₆+0.5 wt % additive according to the invention (a compound of Serie B)) versus Reference at 45° C.

FIG. 6: Nyquist plots of LMFP-LTO battery (PC/EMC/DMC (4/3/3)+1M LiPF₆+0.5 wt % additive according to the invention (a compound of Serie B)) versus Reference, at 0 and 200 cycles.

FIG. 7: Cycling data of LMFP-LTO battery (PC/EMC/DMC (4/3/3)+1M LiPF₆+0.5 wt % additive according to the invention (a compounds of Serie C)) versus Reference after 300 cycles at 45° C.

FIG. 8: Static capacity (0.05C) of LMFP-LTO battery (PC/EMC/DMC (4/3/3)+1M LiPF₆+0.5 wt % additive according to the invention (a compound of Serie C)) versus Reference at 45° C.

FIG. 9: Nyquist plots of LMFP-LTO battery (PC/EMC/DMC (4/3/3)+1M LiPF₆+0.5 wt % additive according to the invention (a compound of Serie C)) versus Reference, at 0 and 100 cycles.

FIG. 10: Cycling data of LMFP-LTO battery (PC/EMC/DMC (4/3/3)+1M LiPF₆+0.5 wt % additive according to the invention (a compounds of Serie D)) versus Reference after 100 cycles at 45° C.

FIG. 11: Static capacity (0.05C) of LMFP-LTO battery (PC/EMC/DMC (4/3/3)+1M LiPF₆+0.5 wt % additive according to the invention (a compound of Serie D)) versus Reference at 45° C.

FIG. 12: a) Cycling performance of NMC 811 half-cells containing additives; b) Magnification of the critical section.

FIG. 13: a) Coulombic efficiency (%) of NMC 811 half-cells containing additives; b) Magnification of the critical section.

FIG. 14: a) Coulombic efficiency (%) of NMC 811 half-cells containing additives; b) Average voltage of NMC 811 half-cells containing additives.

FIG. 15: a) EIS of NMC 811 half-cells containing red-sphere: 3,4-Dimethoxybenzonitrile and blue sphere: reference; b) RCT.

FIG. 16: High-resolution X-ray photoelectron spectra of the cathode after cycling a-c) C 1s, O 1s, and Li 1s with the reference electrolyte; d-f) C 1s, O 1s, and Li 1s with 3,4-dimethoxybenzonitrile additive.

FIG. 17: XPS spectra of N 1s for the cathode after cycling with and without the 3,4-dimethoxybenzonitrile additive.

FIG. 18: Bright-field TEM images of the SEI layer formed at the surface of the NCM after cycling a-d) with the reference electrolyte; e-h) with the 3,4-dimethoxybenzonitrile additive.

FIG. 19: Equivalent Circuit. R1: bulk resistance; R2 and C2: SEI resistance and capacitance; R3 and C3: charge-transfer resistance (Rct) and double-layer capacitance; W: diffusional effects.

FIG. 20: Nyquist plot of the measured EIS for the reference electrolyte at different cycles. Spheres [symbol] represent the experimental data, and square [symbol] s indicate the fitted values.

FIG. 21: Nyquist plot of the measured EIS for the electrolyte containing 0.1 wt % toluene at different cycles. Spheres represent [symbol] the experimental data, while squares [symbol] indicate the fitted values.

FIG. 22: Nyquist plot of the measured EIS for the electrolyte containing 0.1 wt % veratrole at different cycles. Spheres represent [symbol] the experimental data, while squares [symbol] indicate the fitted values.

FIG. 23: Nyquist plot of the measured EIS for the electrolyte containing 0.1 wt % benzonitrile at different cycles. Spheres represent [symbol] the experimental data, while squares [symbol] indicate the fitted values.

FIG. 24: Nyquist plot of the measured EIS for the electrolyte containing 0.1 wt % 1,2,4,5-Benzenetetracarbonitrile at different cycles. Spheres represent [symbol] the experimental data, while squares [symbol] indicate the fitted values.

FIG. 25: Nyquist plot of the measured EIS for the electrolyte containing 0.1 wt % 3,4-Dimethoxybenzonitrile at different cycles. Spheres represent [symbol] the experimental data, while squares [symbol] indicate the fitted values.

### DESCRIPTION OF ILLUSTRATIVE EMBODIMENTS

Before the present invention is further described, it is to be understood that the invention is not limited to the particular embodiments described below, as variations of these embodiments may be made and still fall within the scope of the appended claims. It is also to be understood that the terminology employed is for the purpose of describing particular embodiments, and is not intended to be limiting. Instead, the scope of the present invention will be established by the appended claims.

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

The use of the word “a” or “an” when used in conjunction with the term “comprising” in the claims and/or the specification may mean “one,” but it is also consistent with the meaning of “one or more,” “at least one,” and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein when referring to numerical values or percentages, the term “about” includes variations due to the methods used to determine the values or percentages, statistical variance, and human error. Moreover, each numerical parameter in this application should at least be construed in light of the number of reported significant digits and by applying ordinary rounding techniques.

Term “alkyl” or “alk” as used herein, represents a monovalent group derived from a straight or branched chain saturated hydrocarbon comprising, unless otherwise specified, from 1 to 15 carbon atoms and is exemplified by methyl, ethyl, n-and iso-propyl, n-, sec-, iso-and tert-butyl, neopentyl and the like and may be optionally substituted with one, two, three or, in the case of alkyl groups comprising two carbons or more, four substituents.

The term “alkoxy” or “alkyloxy” as used interchangeably herein, represents an alkyl group attached to the parent molecular group through an oxygen atom.

The term “alkylthio” or “thioalkoxy” as used interchangeably herein, represents an alkyl group attached to the parent molecular group through a sulfur atom.

The term “alkylene” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkenyl” as used herein, represents monovalent straight or branched chain groups of, unless otherwise specified, from 2 to 15 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three or four substituents.

The term “alkynyl” as used herein, represents monovalent straight or branched chain groups of from two to six carbon atoms comprising a carbon-carbon triple bond and is exemplified by ethynyl, 1-propynyl, and the like and may be optionally substituted with one, two, three or four substituents.

The term “cycloalkyl” as used herein, represents a monovalent saturated or unsaturated non-aromatic cyclic hydrocarbon group of three to eight carbon atoms, unless otherwise specified, and is exemplified by cyclopropyl, cyclobutyl, cyclopentyl, cyclohexyl, cycloheptyl, bicyclo[2.2.1]heptyl and the like.

The term “halogen” or “halo” as used interchangeably herein, represents F, Cl, Br, and 1.

The term “heteroatom”, as used herein, is understood as being oxygen, sulfur, or nitrogen.

The inventors have designed and prepared additives for use in association with the electrolyte in a Li-ion battery. The additives of the invention are organic compounds as described herein and which comprise at least one nitrile group. Also, the organic compounds are compatible with the electrolyte as well as other components of the battery.

More specifically, the additives of the invention for use in association with the electrolyte is one of a nitrile-based organic compound as described herein and having general formulae I-XI, A, B, C and D depicted below.

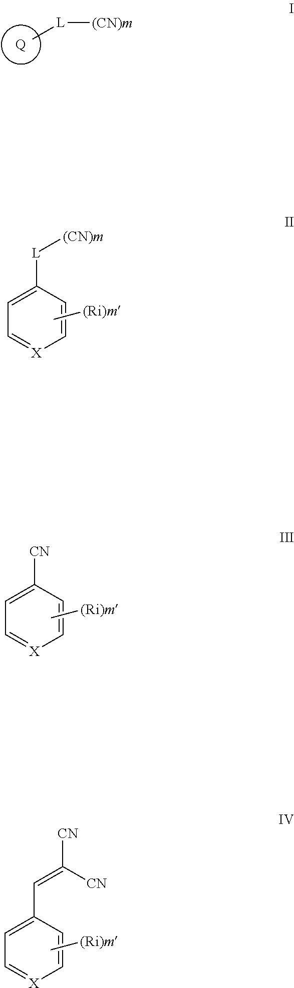

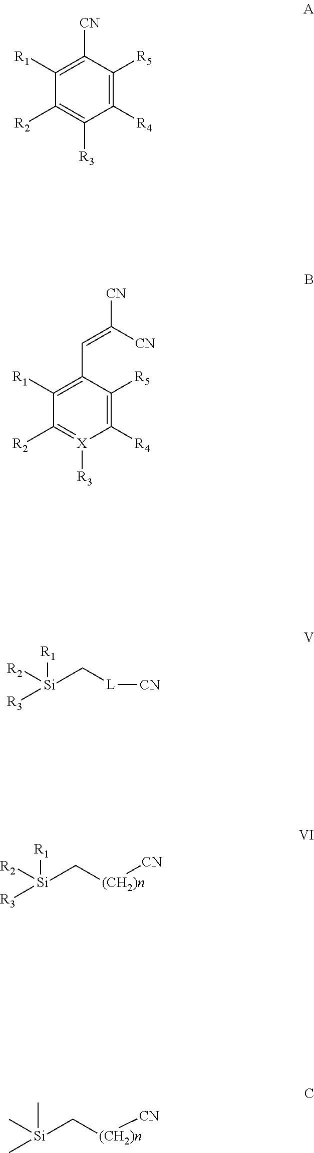

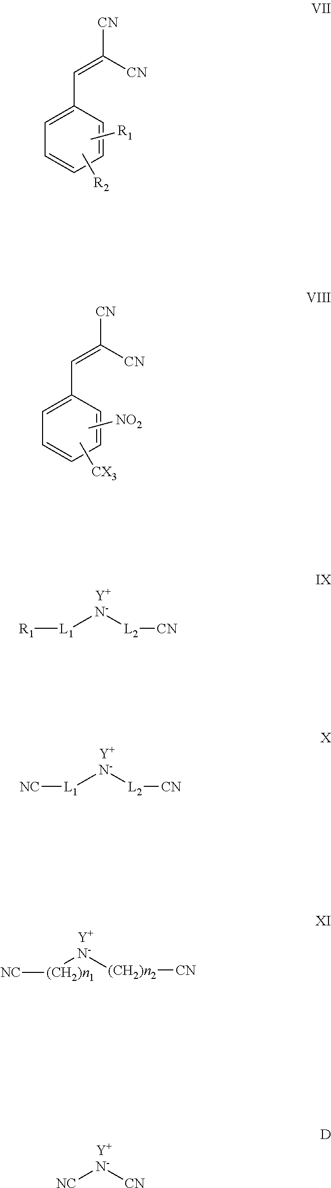

Such organic compounds are exemplified by compounds defined in Table 1 below, namely, Compounds A1-A4, B1-B8, C1-C2 and D1.

TABLE 1
Organic compounds according to the invention (Series A, B, C and D)

| | | R₁ | R₂ | R₃ | R₄ | R₅ | X | n | | Cycle |
|---|---|---|---|---|---|---|---|---|---|---|
| A | A1 | H | H | OMe | OMe | H | | | 0.1% | 300 |
| | A2 | F | F | F | F | F | | | 0.1% | 300 |
| | A3 | F | F | CN | F | F | | | 0.1% | 300 |
| | A4 | CN | H | CN | CN | H | | | 0.1% | 300 |
| B | B1 | H | H | OMe | OMe | H | C | | 0.5% | 300 |
| | B2 | F | F | F | F | F | C | | 0.5% | 200 |
| | B3 | H | H | NO₂ | H | NO₂ | C | | 0.5% | poor results |
| | B4 | H | H | CF₃ | H | NO₂ | C | | 0.5% | 300 |
| | B5 | H | H | CF₃ | H | H | C | | 0.5% | 300 |
| | B6 | H | H | CN | H | H | C | | 0.5% | 300 |
| | B7 | H | H | NO₂ | H | H | C | | 0.5% | poor results |
| | B8 | H | H | H | H | H | N | | 0.5% | 300 |
| C | C1 | | | | | | | 0 | 0.5% | 300 |
| | C2 | | | | | | | 1 | 0.5% | 300 |
| D | D1 | | | | | | | | 0.5% | 300 |

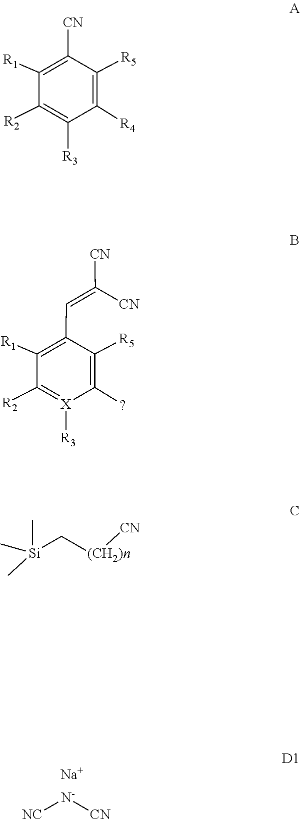

The present invention is illustrated in further details by the following non-limiting examples.

Nitrile-Based Organic Compounds for Use as Additive in Association with Li-Ion Electrolytes

Example 1—General procedure for the preparation of the compounds. To a solution of aldehyde (1 eq.) in 15 mL of chloroform are added, molonodinitrile (1.5 eq.) and few drops of triethylamine. The mixture is refluxed one night under nitrogen. After return to room temperature, dichloromethane is added, and the solution is washed twice with water and dried over MgSO₄. After solvent removal, the residue is chromatographed (silica gel/dichloromethane) to give a solid.

### Example 2—Compound B1

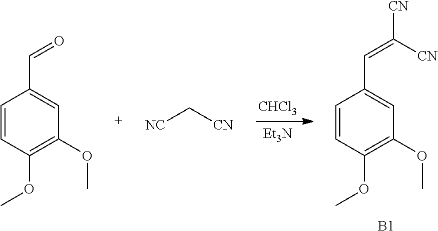

Bright yellow solid (70%). NMR ¹H (400 MHZ, CDCl₃) □: 7.69 (d, 1H, J=4 Hz); 7.64 (s, 1H); 7.38 (dd, 1H, J=4 Hz, J=12 Hz); 6.95 (d, 1H, J=12 Hz); 3.99 (s, 3H); 3.93 (s, 3H).

### Example 3—Compound B2

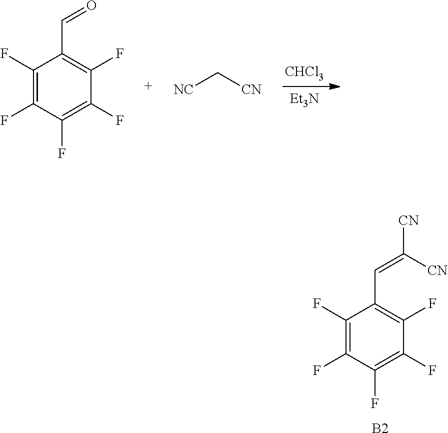

Yellow solid (40%). NMR ¹H (400 MHZ, CDCl₃): 7.77 (s, 1H). NMR ¹⁹F (400 MHz, CDCl₃) □: −132.55 (s, 2H); −143.68 (s, 1H); −158.50 (s, 1H).

### Example 4—Compound B3

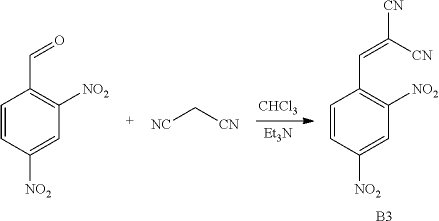

White solid. NMR ¹H (400 MHZ, CDCl₃) □: 8.60 (d, 1H, J=4 Hz); 8.25 (dd, 1H, J=4 Hz, J=12 Hz); 8.18 (s, 1H); 8.15 (d, 1H, J=12 Hz).

### Example 5—Compound B4

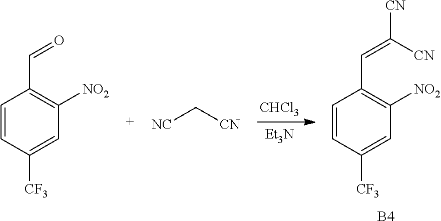

Bright yellow solid. NMR ¹H (400 MHZ, CDCl₃) □: 8.12 (d, 1H, J=4 Hz); 8.03 (s, 1H); 7.67 (dd, 1H, J=4 Hz, J=12 Hz). NMR ¹⁹F (400 MHZ, CDCl₃) □: −63.65 (s, 3F).

### Example 6—Compound B5

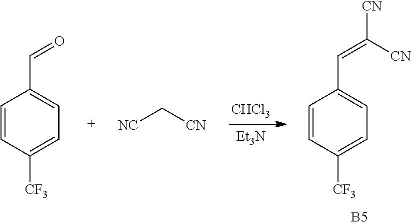

White solid. NMR ¹H (400 MHZ, CDCl₃) □: 8.02 (d, 2H, J=12 Hz); 7.83 (d, 2H, J=8 Hz); 7.80 (s, 1H). NMR ¹⁹F (400 MHZ, CDCl₃) □: −63.48 (s, 3F).

### Example 7—Compound B6

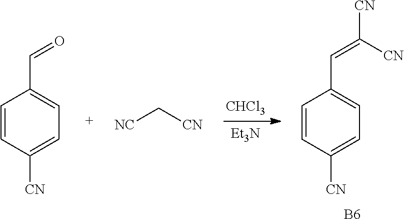

White solid. NMR ¹H (400 MHZ, CDCl₃) □: 7.99 (d, 2H, J=8 Hz); 7.83 (d, 2H, J=8 Hz); 7.74 (s, 1H).

### Example 8—Compound B7

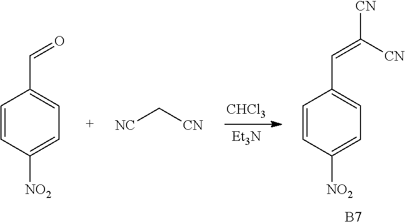

Pale orange solid. NMR ¹H (400 MHZ, CDCl₃) □: 8.39 (d, 2H, J=12 Hz); 8.07 (d, 2H, J=8 Hz); 7.88 (s, 1H).

### Example 9—Compound B8

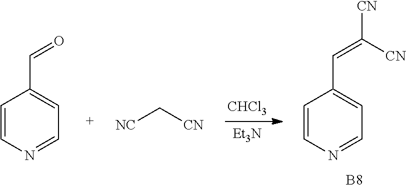

Pink solid. NMR ¹H (400 MHZ, CDCl₃) □: 8.89 (d, 2H, J=12 Hz); 7.81 (s, 2H); 7.68 (d, 2H, J=8 Hz).

Compounds of the Series A and C and Compound D1 are commercially available and were used as received.

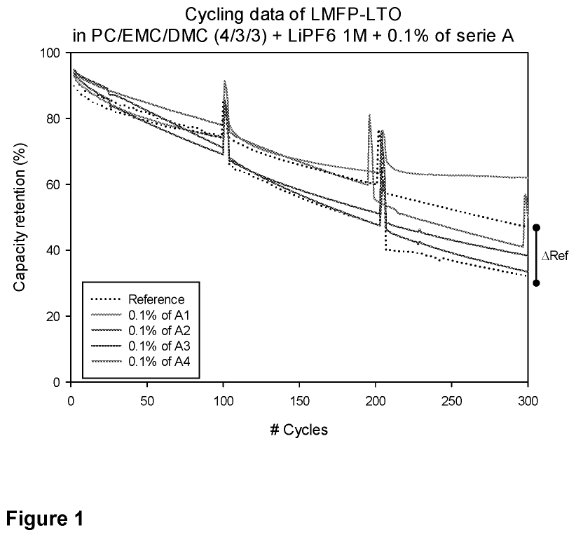

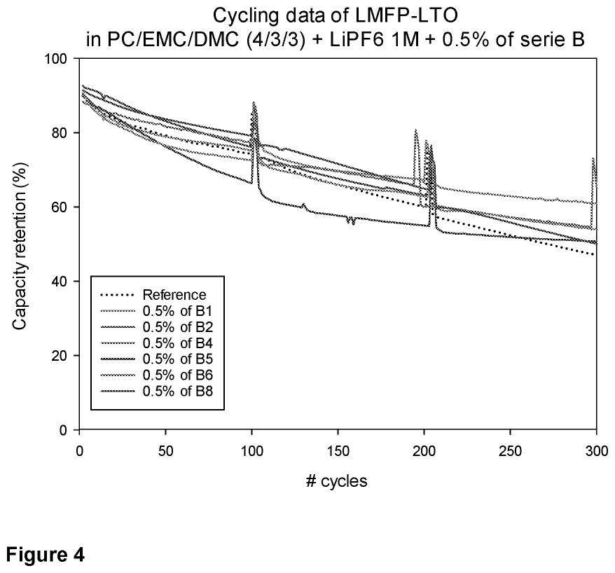

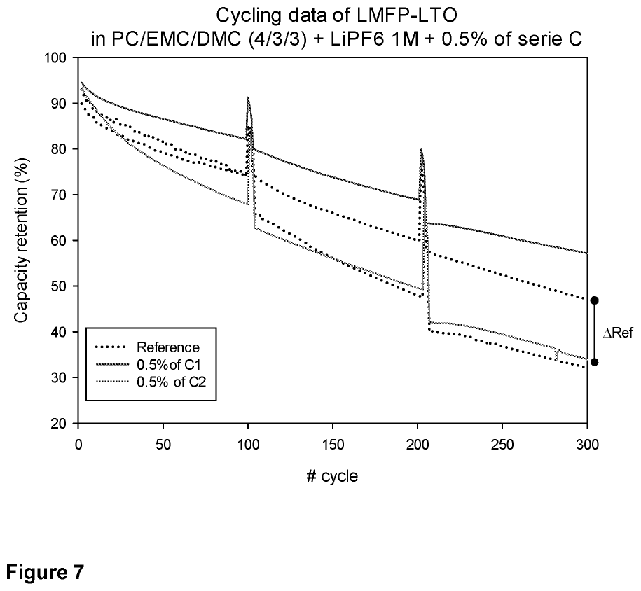

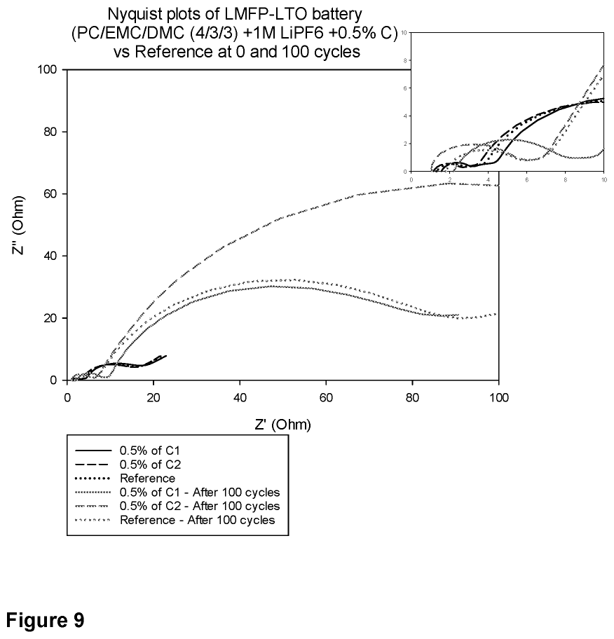

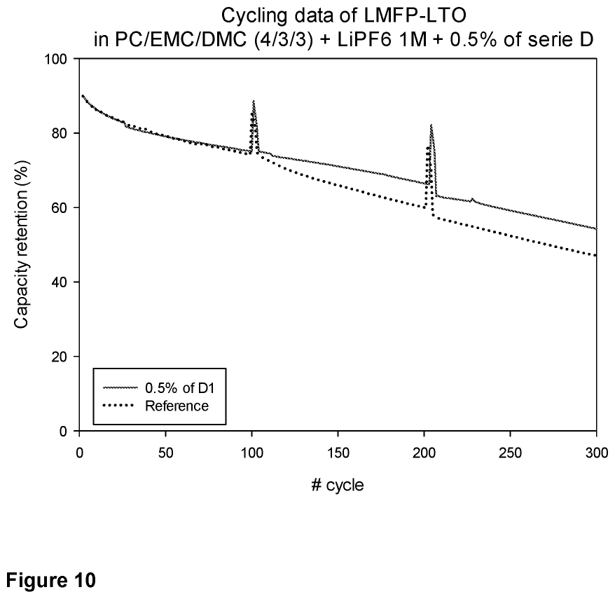

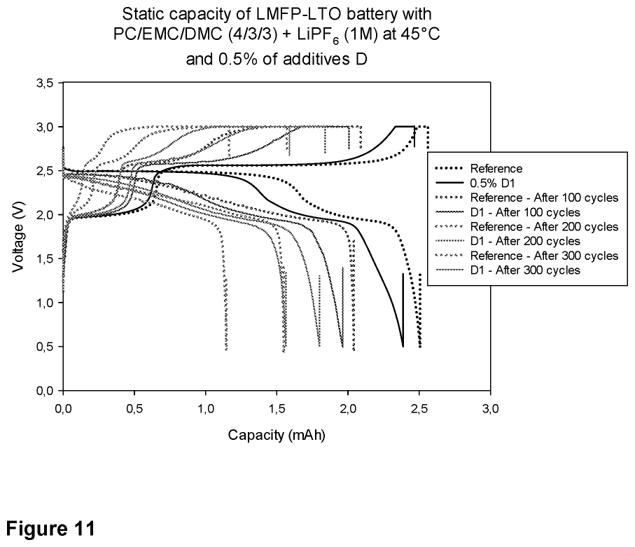

Referring to the figures, FIGS. 1-3 outline results obtained using compounds of the Serie A; FIGS. 4-6 outline results obtained using compounds of the Serie B; FIGS. 7-9 outline results obtained using compounds of the Serie C; and FIGS. 10-11 outline results obtained using compounds of the Serie D. It should be noted that Reference batteries as well as batteries according to the invention, do not contain vinylene carbonate (VC), which explains the poor stability after 300 cycles. Nonetheless as can be seen, batteries comprising the additive according to the invention present a far better stability.

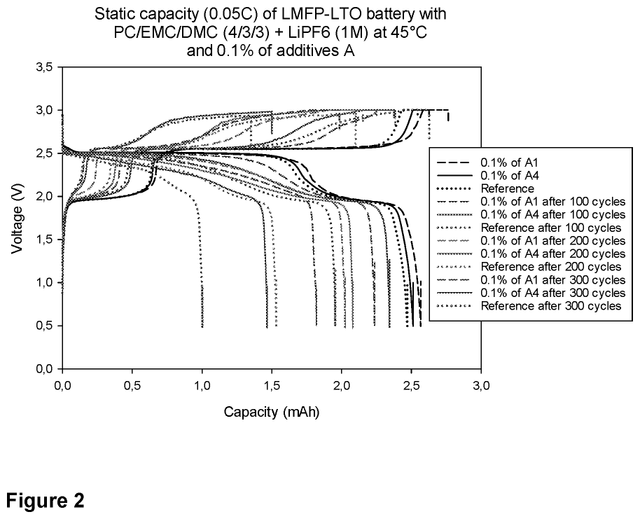

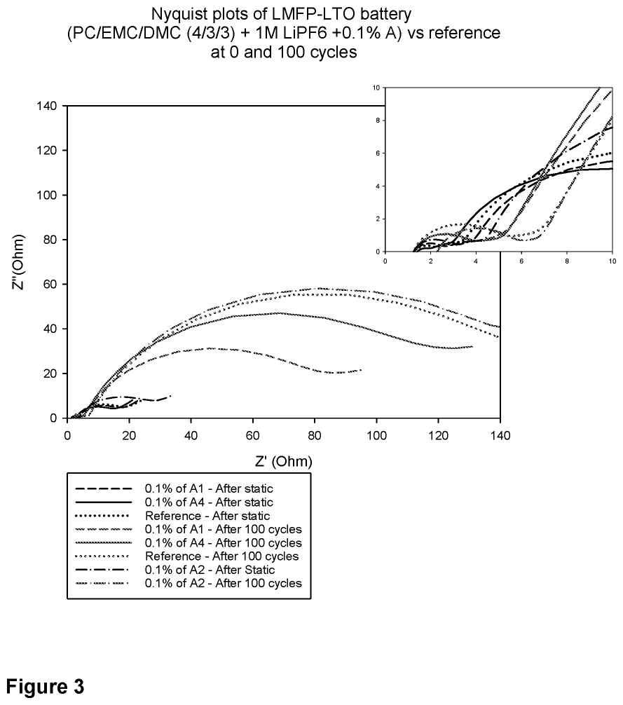

As can be seen in FIG. 2, use of 0.1 wt % of compound A1 or A4 allows for improvement of the battery capacity as well as a better reversibility. Moreover, a global decrease of the battery resistance is noted (FIG. 3).

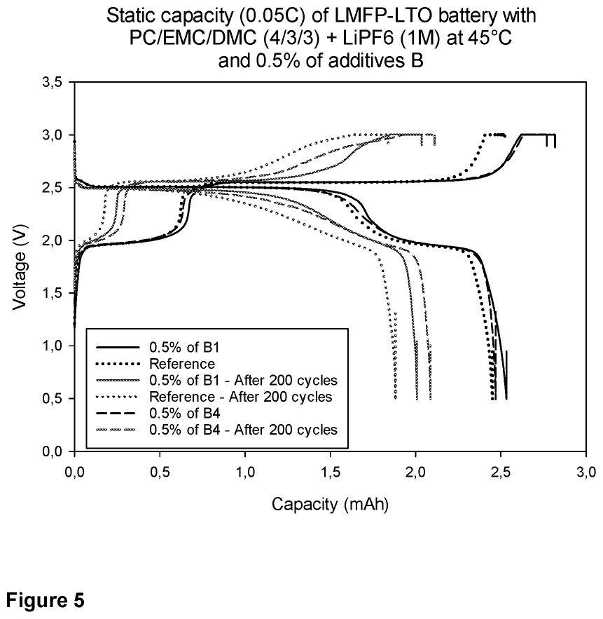

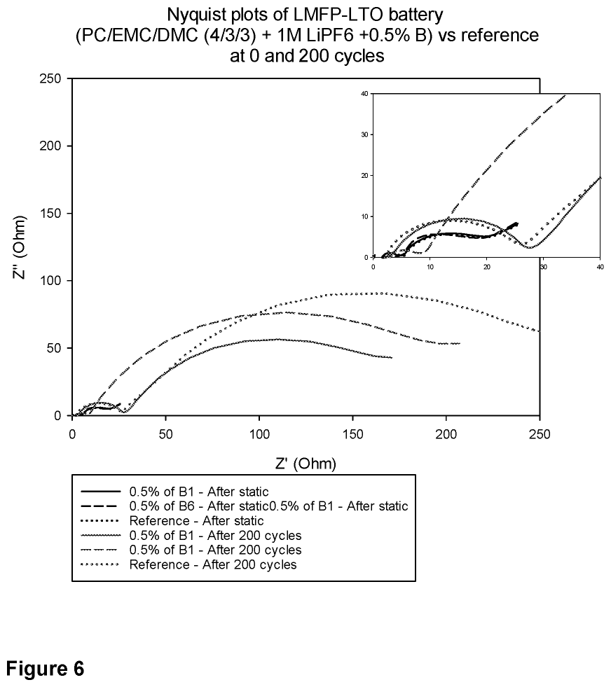

FIG. 5 shows results obtained for compounds B1 and B4. Use of 0.5 wt % of the additive allows for an improvement of the battery capacity. A global decrease of the battery resistance is noted (FIG. 6).

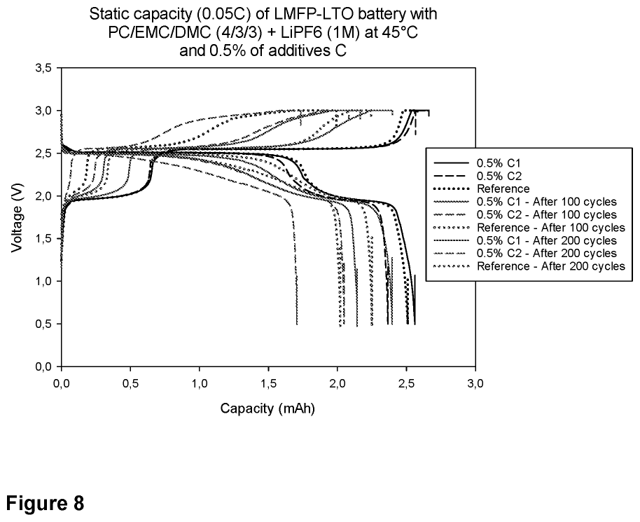

FIG. 7 shows results obtained for compounds C1 and C2. Use of 0.5 wt % of the additive yields a good stability after 300 cycles at 45° C. As can be seen in FIG. 8, better results are obtained for compound C1 (shorter carbon chain).

FIG. 10 shows results obtained for compound D1. As can be seen in FIG. 11, use of 0.5 wt % of compound D1 allows for improvement of the battery capacity as well as a better reversibility.

In an embodiment, 3,4-dimethoxybenzonitrile is used as an additive to create a stable cathode electrolyte interphase (CEI), while vinylene carbonate (VC) is retained as the main additive in a standard electrolyte consisting of LiPF₆ and a carbonate solution. Initially considered for their redox-shuffling capabilities, dimethoxybenzene and its derivatives [23,24] have also been proposed as agents for trapping manganese [25]. This approach helps mitigate the issue of manganese dissolution. In embodiments described here, 3,4-dimethoxybenzene and benzonitrile were evaluated in lithium battery systems. These tests demonstrated that the methoxy and nitrile groups exhibit a synergistic effect, enhancing overall performance and providing a robust cathode-electrolyte interphase (CEI). All experiments were carried out using a Li∥NMC811 cell architecture. These embodiments underscore the role of functional group engineering for optimizing electrolyte additives with high-nickel NMC.

### Electrodes Preparation

The composite electrode was made with NMC 811 (single crystal from BASF), carbon black (Denka-black), carbon nanofiber (Tuball Battery-grade) and polyvinylidene fluoride (PVDF) binder (KF #9700) was mixed in N-methylpyrrolidone (99%, extra pure, Thermo Scientific Chemicals) in a Dry-room with a dew point of −60° C. The slurry was coated on an ~17 μm aluminum current collector. The electrodes were dried under vacuum during at least 12h. at 120° C. The average loading is 11.2 mg/cm² in active materials. The electrode formulation consists of 96.5 wt % NCM, 3.0 wt % PVdF binder, 0.4 wt % Denka Black, and 0.1 wt % carbon nanotubes (CNT).

### Electrolyte Preparation

Ethylene carbonate (EC), ethyl methyl carbonate (EMC), dimethyl carbonate (DMC) and vinylene carbonate (VC) are sourced from Gotion, while lithium hexafluorophosphate (LiPF₆) is sourced from BASF. 1M LiPF₆ EC:EMC:DMC (4:3:3 in volumetric ratio) +2% VC (mass ratio) was used as reference electrolyte. From this reference, 0.1% (mass ratio) of additive has been added. Table 2 presents the list of additives used, while Table 3 shows their chemical structures. All additives except toluene were dried at 60° C. under vacuum prior to use.

TABLE 2
Chemical names and sources of the additives used

| Name | Source |
|---|---|
| Toluene | Sigma Aldrich, 99.8%, anhydrous |
| Benzonitrile | TCI, 99+% |
| 3,4-Dimethoxybenzonitrile* | Sigma Aldrich ≥99% |
| 1,2-Dimethoxybenzene(veratrole) | TCI, 99+% |
| 1,2,4,5-Benzenetetracarbonitrile* | Sigma Aldrich 97% |

*The molecules 3,4-dimethoxybenzonitrile and 1,2,4,5-benzenetetracarbonitrile used in this study are subject to existing patents.²³
To simplify the text, 1,2,4,5-Benzenetetracarbonitrile will be referred as Benzenetetracarbonitrile.

TABLE 3
EIS fitting data for Reference - 50 Cycles

| | | | |
|---|---|---|---|
| Parameter | Value | Std. Error | Rank |

| | | | | | |
|---|---|---|---|---|---|
| R1 | 3.73 | Ω | 0.045 | Ω | 1 |
| C2 | 71.04 | μF | 4.09 | μF | 0.417 |
| R2 | 1.95 | Ω | 0.045 | Ω | 0.720 |
| C3 | 1.93 | mF | 54.41 | μF | 0.491 |
| R3 | 3.40 | Ω | 0.031 | Ω | 0.571 |
| s3 | 2.20 | Ω · s^{-1/2} | 0.031 | Ω · s^{-1/2} | 0.301 |

Chi²/|Z|² = 0.092;
Chi/√N = 0.16 Ω

### Electrochemical Test

CR2032 coin cells were assembled in an argon-filled glovebox using separators supplied by W-Scope. The anode consisted of lithium metal (200 μm thick, 99.9% purity) sourced from China Energy Lithium Co. Ltd. All electrochemical measurements, including galvanostatic cycling and electrochemical impedance spectroscopy (EIS), were conducted at 25° C. in a temperature-controlled environmental chamber. A Biologic BCS-series potentiostat was used for all tests.

The cycling protocol used is as follows: cells were cycled between 3.0 V and 4.3 V. Each charge step included a constant voltage (CV) step, held until the current reached C/20 or for a maximum time of 1 hour. Two initial formation cycles were performed at C/10 for both charge and discharge. This was followed by 48 cycles at rates ranging from C/3 to 1C. Finally, two additional cycles at C/10 were carried out, during which EIS measurements were performed after charging and a 2-hour rest period. EIS spectra were recorded in the frequency range of 0.01 Hz to 500 mHz with a perturbation amplitude of 5.0 mV.

### Ex-Situ Characterization

Post-mortem analyses were carried out on two sets of lithium-ion batteries: one employing the reference electrolyte and the other containing 3,4-Dimethoxybenzonitrile as an additive. The cells were interrupted after the discharge phase after 50 cycles. Prior to characterization, the electrodes were carefully rinsed with dimethyl carbonate to eliminate residual electrolyte.

XPS measurements were performed using a PHI VersaProbe III spectrometer equipped with a monochromatic Al Kα X-ray source (hv=1486.6 eV). The source was operated at 25 W with a spot size of 100 μm. Samples were mounted on the sample holder inside a glove box and transferred to the XPS instrument using a sealed transfer vessel to maintain an inert atmosphere. Survey spectra were collected at a pass energy of 224 eV, while high-resolution spectra were acquired at 55 eV. Data processing was carried out using CasaXPS software [27]. A Shirley background was subtracted, and peaks were fitted using a Gaussian-Lorentzian product function (GL (30)). Binding energies were calibrated against the C 1s peak at 284.8 eV.

Sample preparation for TEM imaging was performed by gently scraping the cathode material from the aluminum current collector using a spatula. The collected material was dispersed in dimethyl carbonate (DMC) and sonicated for 5 minutes. After resting for 1 minute, the supernatant was drop-cast onto a lacey carbon-coated copper TEM grid. TEM observations were carried out using a Hitachi HF3300 cold field-emission TEM operated at an accelerating voltage of 300 kV. Bright-field images were acquired with a Gatan 994 UltraScan 1000XP camera.

### Optimization of Additive Choice Through Functional Group Analysis

In the present invention, a range of additives with diverse functional groups were investigated to assess their influence on lithium battery cyclability. The first group of compounds includes benzonitrile and benzenetetracarbonitrile, both featuring nitrile-substituted benzene rings. The second group comprises veratrole, which introduces methoxy functionalities, and toluene, used as a reference compound representing a simple benzyl group. Finally, 3,4-dimethoxybenzonitrile was chosen as a multifunctional additive, combining benzyl, nitrile, and methoxy groups within a single molecular structure. To establish a baseline for comparison, a reference electrolyte (1M LiPF₆ EC:EMC:DMC (4:3:3 in volumetric ratio)+2% VC (mass ratio) was also tested. This selection enables a comparative analysis of the impact of individual and combined functional groups on battery performance.

### Impact of Additives on Cycling Performance

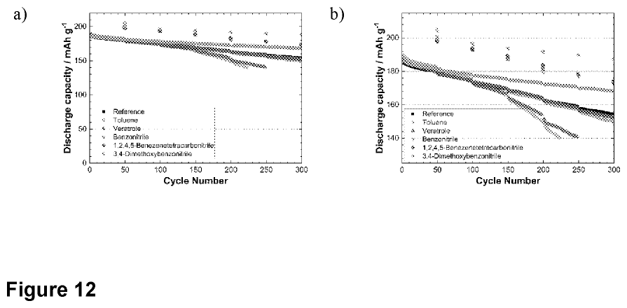

The initial test was carried out on a series of coin cells with 0.1 wt % of additive (FIGS. 12a and 12b). The first observation is that the discharge capacity at 1C is not significantly affected by the additives, with a highest capacity of 189.8 mAh/g with the Toluene additive, and a lowest of 185.8 mAh/g for the reference sample—a difference of approximately 2%. Although this variation in capacity is relatively modest, it indicates that the additives do not significantly hinder capacities in the early cycles. The primary benefit of these additives, however, lies in their ability to enhance long-term performance.

In this regard, the analysis of capacity retention reveals a significant impact of the additives after ~50 cycles. The achievement of 80% capacity retention varies considerably depending on the additive: 350 cycles for the reference, 192 cycles for Toluene, 204 cycles for Veratrole, 301 cycles for Benzenetetracarbonitrile, and 288 cycles for Benzonitrile. Notably, 3,4-Dimethoxybenzonitrile retained 89% of its initial capacity at 1C even after 334 cycles, demonstrating superior long-term stability.

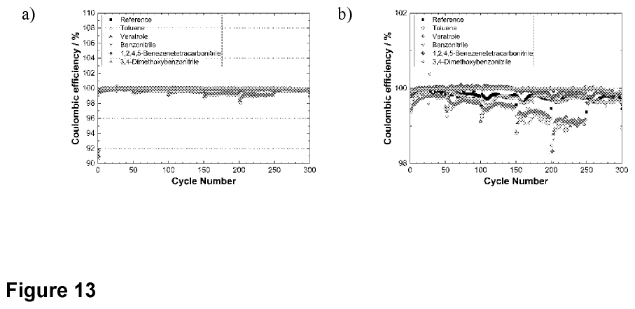

The importance of coulombic efficiency is well established [28], especially when the lithium source could be a limiting factor [29]. FIGS. 13a and 13b show the coulombic efficiency as a function of the number of cycles. For toluene and veratrole, the values range between 99.4% and 99.8% during the first 100 cycles, then quickly drop to 99%. With the benzonitrile additive, the first 50 cycles are around 99.98%, while for the reference, the values typically range between 99.7% and 99.8%. The benzenetetracarbonitrile additive maintains a value close to unity from cycles 6 to 150, then decreases to around 99.8% thereafter. Finally, the 3,4-dimethoxybenzonitrile additive provides an average of 99.97% from cycle 5 to 300.

TABLE 4
Coulombic efficiency (%) as a function of cycle number

cycle number

| Name | 1 | 100 | 200 | 300 |
|---|---|---|---|---|
| Reference | 91.56 | 99.54 | 99.46 | 99.46 |
| Toluene | 90.72 | 99.14 | 98.48 | n/a |
| Veratrole | 90.99 | 99.12 | 98.30 | n/a |
| Benzonitrile | 91.70 | 99.17 | 99.25 | n/a |
| 1,2,4,5- | 91.70 | 100.02 | 99.68 | 99.58 |
| Benzenetetracarbonitrile | | | | |
| 3,4-Dimethoxybenzonitrile | 91.67 | 100.01 | 99.97 | 99.77 |

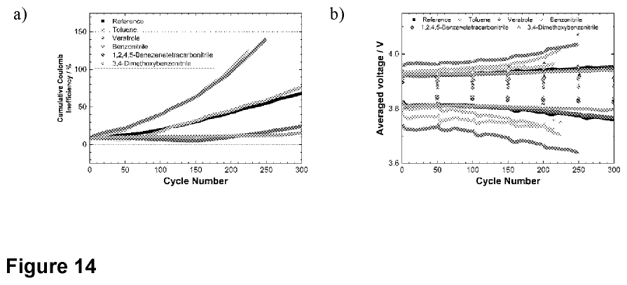

Cumulative coulombic inefficiency (FIG. 14) is another useful metric for representing coulombic efficiency over extended cycling. After 200 cycles, the cumulative inefficiency values are as follows: Reference (42.6%), Toluene (101%), Veratrole (97.0%), Benzonitrile (49.8%), Benzenetetracarbonitrile (10.2%), and 3,4-Dimethoxybenzonitrile (11.7%). These results highlight the superior performance of Benzenetetracarbonitrile and 3,4-Dimethoxybenzonitrile in minimizing irreversible losses over long-term cycling.

The evolution of internal resistance in the cell serves as a key indicator of the influence of additives, providing insights into the mechanisms governing the growth or stabilization of interfacial layers such as the cathode-electrolyte interphase (CEI) and the solid-electrolyte interphase (SEI). The average potential during charge and discharge cycles emerges as a relevant comparative parameter, reflecting the impact of additives on the electrochemical behavior and resistance development within the cell. In FIG. 14b, the average potential is plotted as a function of the number of cycles. It can be observed that the additive veratrole exhibits higher resistance from the very first cycles, whereas resistance develops more with cycles with benzonitrile, followed by toluene. This effect is more significant in the discharge phase compared to the charge phase. In contrast, for the reference, Benzenetetracarbonitrile, and 3,4-Dimethoxybenzonitrile, the average potential remains similar for at least the first 150 cycles on discharge and 250 cycles on charge. It can therefore be concluded that the layer formed by 3,4-Dimethoxybenzonitrile is less resistive than those formed by the other additives and the reference.

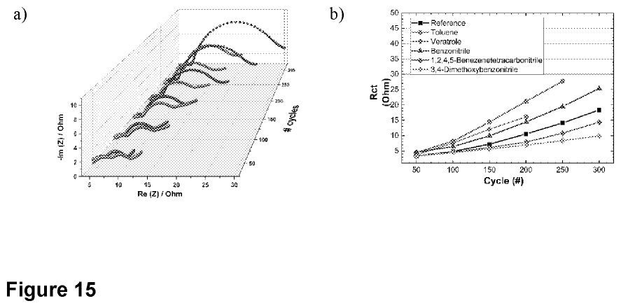

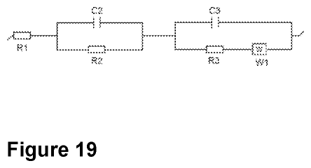

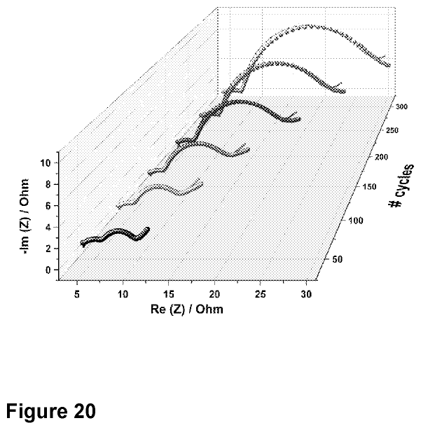

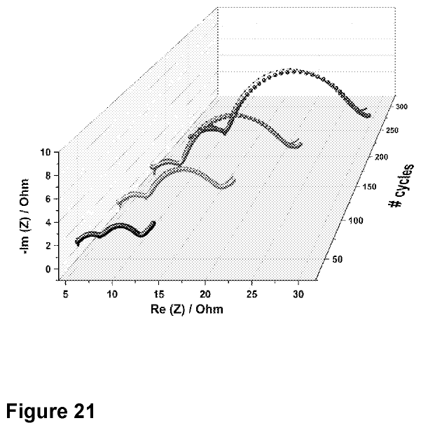

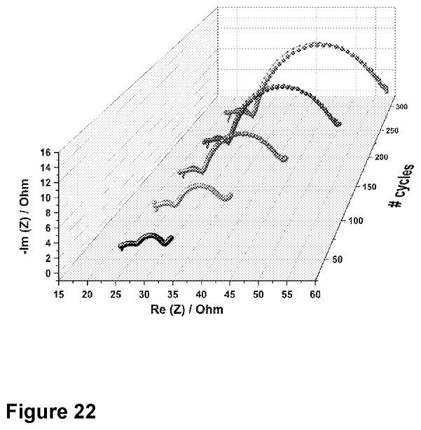

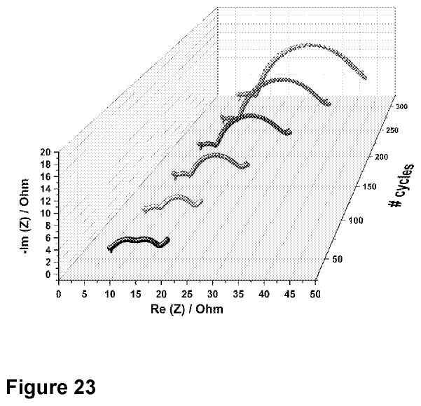

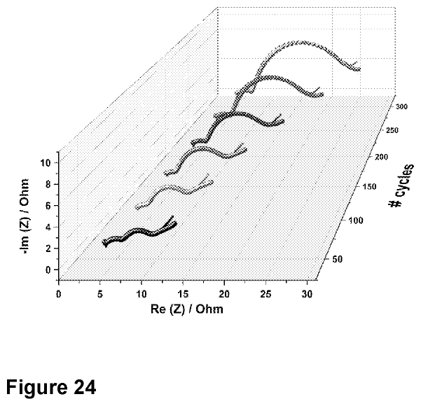

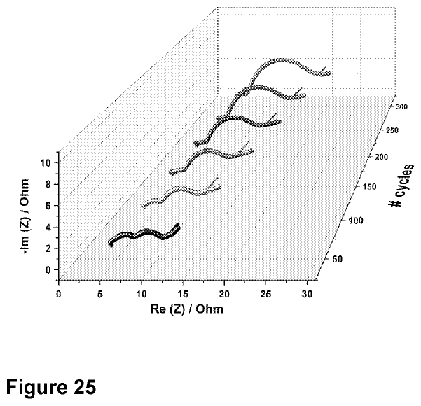

To further understand the resistances in these batteries, Electrochemical Impedance Spectroscopy (EIS) was analyzed. EIS was performed every 50 cycles. In FIG. 15a, the EIS is compared between the reference system and the one with 3,4-Dimethoxybenzonitrile. It can be observed that the behavior is similar, but the Rct component of the 3,4-Dimethoxybenzonitrile system is smaller, particularly from cycle 150 onward. All EIS spectra are shown in FIGS. 20-25. To deepen the analysis, a Randles circuit (FIG. 19) was used to fit all the EIS data. The results of all these fits are reported in Table 3 above and Tables 5-37 below. FIG. 15b presents the charge transfer resistance (CTR) component for the different additives, compared to the reference system, as a function of the cycle number. It can be observed that the Rct is higher for batteries containing the additives benzonitrile, toluene and veratrole compared to the reference. While those with Benzenetetracarbonitrile and 3,4-Dimethoxybenzonitrile have a lower Rct than the reference.

This variation in charge transfer resistance may be explained by the effect of the additive on the quality of the cathode electrolyte interphase (CEI) layer. Further material characterization can be performed.

TABLE 5
EIS fitting data for Reference - 100 Cycles

| | | | |
|---|---|---|---|
| Parameter | Value | Std. Error | Rank |

| | | | | | |
|---|---|---|---|---|---|
| R1 | 4.08 | Ω | 0.043 | Ω | 1 |
| C2 | 78.67 | μF | 4.88 | μF | 0.441 |
| R2 | 1.75 | Ω | 0.045 | Ω | 0.708 |
| C3 | 2.07 | mF | 39.00 | μF | 0.559 |
| R3 | 4.85 | Ω | 0.042 | Ω | 0.645 |
| s3 | 3.55 | Ω · s^{-1/2} | 0.090 | Ω · s^{-1/2} | 0.336 |

Chi²/|Z|² = 0.090;
Chi/√N = 0.2071 Ω

TABLE 6
EIS fitting data for Reference - 150 Cycles

| | | | |
|---|---|---|---|
| Parameter | Value | Std. Error | Rank |

| | | | | | |
|---|---|---|---|---|---|
| R1 | 4.42 | Ω | 0.050 | Ω | 1 |
| C2 | 76.36 | μF | 5.29 | μF | 0.450 |
| R2 | 1.77 | Ω | 0.049 | Ω | 0.697 |
| C3 | 2.20 | mF | 35.42 | μF | 0.597 |
| R3 | 7.21 | Ω | 0.059 | Ω | 0.644 |
| s3 | 3.89 | Ω · s^{-1/2} | 0.12 | Ω · s^{-1/2} | 0.330 |

Chi²/|Z|² = 0.106;
Chi/√N = 0.2537 Ω

TABLE 7
EIS fitting data for Reference - 200 Cycles

| | | | |
|---|---|---|---|
| Parameter | Value | Std. Error | Rank |

| | | | | | |
|---|---|---|---|---|---|
| R1 | 4.80 | Ω | 0.064 | Ω | 1 |
| C2 | 60.64 | μF | 4.21 | μF | 0.467 |
| R2 | 2.2 | Ω | 0.063 | Ω | 0.713 |
| C3 | 2.22 | mF | 35.04 | μF | 0.637 |
| R3 | 10.55 | Ω | 0.089 | Ω | 0.641 |
| s3 | 4.21 | Ω · s^{-1/2} | 0.16 | Ω · s^{-1/2} | 0.317 |

Chi²/|Z|² = 0.1445;
Chi/√N = 0.3412 Ω

TABLE 8
EIS fitting data for Reference - 250 Cycles

| | | | |
|---|---|---|---|
| Parameter | Value | Std. Error | Rank |

| | | | | | |
|---|---|---|---|---|---|
| R1 | 5.08 | Ω | 0.073 | Ω | 1 |
| C2 | 52.82 | μF | 3.73 | μF | 0.480 |
| R2 | 2.51 | Ω | 0.072 | Ω | 0.720 |
| C3 | 2.23 | mF | 33.61 | μF | 0.675 |
| R3 | 14.13 | Ω | 0.12 | Ω | 0.634 |
| s3 | 4.51 | Ω · s^{-1/2} | 0.21 | Ω · s^{-1/2} | 0.309 |

Chi²/|Z|² = 0.1711;
Chi/√N = 0.4203 Ω

TABLE 9
EIS fitting data for Reference - 300 Cycles

| | | | |
|---|---|---|---|
| Parameter | Value | Std. Error | Rank |

| | | | | | |
|---|---|---|---|---|---|
| R1 | 5.44 | Ω | 0.088 | Ω | 1 |
| C2 | 47.94 | μF | 3.60 | μF | 0.487 |
| R2 | 2.71 | Ω | 0.087 | Ω | 0.721 |
| C3 | 2.24 | mF | 31.47 | μF | 0.704 |
| R3 | 18.34 | Ω | 0.17 | Ω | 0.630 |
| s3 | 4.86 | Ω · s^{-1/2} | 0.27 | Ω · s^{-1/2} | 0.306 |

Chi²/|Z|² = 0.1917;
Chi/√N = 0.4982 Ω

TABLE 10
EIS fitting data for Reference - 350 Cycles

| | | | |
|---|---|---|---|
| Parameter | Value | Std. Error | Rank |

| | | | | | |
|---|---|---|---|---|---|
| R1 | 5.53 | Ω | 0.089 | Ω | 1 |
| C2 | 48.84 | μF | 3.71 | μF | 0.496 |
| R2 | 2.81 | Ω | 0.088 | Ω | 0.722 |
| C3 | 2.23 | mF | 30.03 | μF | 0.736 |
| R3 | 23.01 | Ω | 0.20 | Ω | 0.626 |
| s3 | 5.29 | Ω · s^{-1/2} | 0.34 | Ω · s^{-1/2} | 0.305 |

Chi²/|Z|² = 0.2143;
Chi/√N = 0.5767 Ω

TABLE 11
EIS fitting data for Toluene 50 cycles

| | | | |
|---|---|---|---|
| Parameter | Value | Std. Error | Rank |

| | | | | |
|---|---|---|---|---|
| R1 | 4.35 | Ohm | 0.041 | 1 |
| C2 | 7.2e−05 | F | 3.12e−06 | 0.430 |
| R2 | 2.47 | Ohm | 0.045 | 0.723 |
| C3 | 0.00189 | F | 4.74e−05 | 0.489 |
| R3 | 3.83 | Ohm | 0.031 | 0.562 |
| s3 | 2.43 | Ohm · s\hat{ } − ½ | 0.029 | 0.294 |

Chi²/|Z|² = 0.0843;
Chi/√N = 0.1748 Ω

TABLE 12
EIS fitting data for Toluene 100 cycles

| | | | |
|---|---|---|---|
| Parameter | Value | Std. Error | Rank |

| | | | | |
|---|---|---|---|---|
| R1 | 5.38 | Ohm | 0.063 | 1 |
| C2 | 6.038e−05 | F | 3.07e−06 | 0.470 |
| R2 | 3.18 | Ohm | 0.065 | 0.744 |
| C3 | 0.00216 | F | 4.05e−05 | 0.557 |
| R3 | 7.54 | Ohm | 0.0542 | 0.619 |
| s3 | 3.23 | Ohm · s\hat{ } − ½ | 0.079 | 0.291 |

Chi²/|Z|² = 0.1210;
Chi/√N = 0.2792 Ω

TABLE 13
EIS fitting data for Toluene 150 cycles

| | | | |
|---|---|---|---|
| Parameter | Value | Std. Error | Rank |

| | | | | |
|---|---|---|---|---|
| R1 | 6.095 | Ohm | 0.0816 | 1 |
| C2 | 5.28e−05 | F | 3.193e−06 | 0.476 |
| R2 | 3.39 | Ohm | 0.080 | 0.727 |
| C3 | 0.00223 | F | 3.25e−05 | 0.596 |
| R3 | 12.11 | Ohm | 0.083 | 0.609 |
| s3 | 3.584 | Ohm · s\hat{ } − ½ | 0.127 | 0.280 |

Chi²/|Z|² = 0.1506;
Chi/√N = 0.3674 Ω

TABLE 14
EIS fitting data for Toluene 200 cycles

| | | | |
|---|---|---|---|
| Parameter | Value | Std. Error | Rank |

| | | | | |
|---|---|---|---|---|
| R1 | 7.88 | Ohm | 0.098 | 1 |
| C2 | 4.93e−05 | F | 2.70e−06 | 0.493 |
| R2 | 4.56 | Ohm | 0.098 | 0.739 |
| C3 | 0.0021 | F | 3.15e−05 | 0.611 |
| R3 | 16.15 | Ohm | 0.115 | 0.596 |
| s3 | 3.680 | Ohm · s\hat{ } − ½ | 0.175 | 0.262 |

Chi²/|Z|² = 0.155;
Chi/√N = 0.481 Ω

TABLE 15
EIS fitting data for Veratrole 50 cycles

| | | | |
|---|---|---|---|
| Parameter | Value | Std. Error | Rank |

| | | | | |
|---|---|---|---|---|
| R1 | 23.1 | Ohm | 0.069 | 1 |
| C2 | 6.629e−05 | F | 4.18e−06 | 0.431 |
| R2 | 2.63 | Ohm | 0.0654 | 0.731 |
| C3 | 0.00213 | F | 4.69e−05 | 0.496 |
| R3 | 4.482 | Ohm | 0.035 | 0.557 |
| s3 | 2.147 | Ohm · s\hat{ } − ½ | 0.0247 | 0.283 |

Chi²/|Z|² = 0.00799;
Chi/√N = 0.228 Ω

TABLE 16
EIS fitting data for Veratrole 100 cycles

| | | | |
|---|---|---|---|
| Parameter | Value | Std. Error | Rank |

| | | | | |
|---|---|---|---|---|
| R1 | 23.97 | Ohm | 0.11 | 1 |
| C2 | 4.216e−05 | F | 2.96e−06 | 0.44 |
| R2 | 3.70 | Ohm | 0.105 | 0.72 |
| C3 | 0.00225 | F | 4.213e−05 | 0.531 |
| R3 | 8.18 | Ohm | 0.053 | 0.531 |
| s3 | 3.02 | Ohm · s\hat{ } − ½ | 0.0646 | 0.265 |

Chi²/|Z|² = 0.0183;
Chi/√N = 0.3674 Ω

TABLE 17
EIS fitting data for Veratrole 150 cycles

| | | | |
|---|---|---|---|
| Parameter | Value | Std. Error | Rank |

| | | | | |
|---|---|---|---|---|
| R1 | 24.01 | Ohm | 0.122 | 1 |
| C2 | 3.727e−05 | F | 2.675e−06 | 0.457 |
| R2 | 4.171 | Ohm | 0.119 | 0.707 |
| C3 | 0.00237 | F | 3.219e−05 | 0.569 |
| R3 | 14.45 | Ohm | 0.0834 | 0.51 |
| s3 | 3.323 | Ohm · s\hat{ } − ½ | 0.120 | 0.248 |

Chi²/|Z|² = 0.0267;
Chi/√N = 0.464 Ω

TABLE 18
EIS fitting data for Veratrole 200 cycles

| | | | |
|---|---|---|---|
| Parameter | Value | Std. Error | Rank |

| | | | | |
|---|---|---|---|---|
| R1 | 24.26 | Ohm | 0.152 | 1 |
| C2 | 3.14e−05 | F | 2.40e−06 | 0.460 |
| R2 | 4.81 | Ohm | 0.148 | 0.702 |
| C3 | 0.00239 | F | 2.98e−05 | 0.590 |
| R3 | 21.156 | Ohm | 0.140 | 0.505 |
| s3 | 3.83 | Ohm · s\hat{ } − ½ | 0.207 | 0.241 |

−Chi²/|Z|² = 0.039;
Chi/√N = 0.598 Ω

TABLE 19
EIS fitting data for Veratrole 250 cycles

| | | | |
|---|---|---|---|
| Parameter | Value | Std. Error | Rank |

| | | | | |
|---|---|---|---|---|
| R1 | 24.34 | Ohm | 0.192 | 1 |
| C2 | 2.55e−05 | F | 2.0e−06 | 0.476 |
| R2 | 5.88 | Ohm | 0.190 | 0.716 |
| C3 | 0.00238 | F | 3.197e−05 | 0.611 |
| R3 | 27.67 | Ohm | 0.220 | 0.526 |
| s3 | 4.68 | Ohm · s\hat{ } − ½ | 0.313 | 0.240 |

Chi²/|Z|² = 0.0613;
Chi/√N = 0.785 Ω

TABLE 20
EIS fitting data for Benzonitrile 50 cycles

| | | | |
|---|---|---|---|
| Parameter | Value | Std. Error | Rank |

| | | | | |
|---|---|---|---|---|
| R1 | 6.91 | Ohm | 0.076 | 1 |
| C2 | 7.14e−05 | F | 3.368e−06 | 0.450 |
| R2 | 4.24 | Ohm | 0.091 | 0.736 |
| C3 | 0.00129 | F | 6.14e−05 | 0.481 |
| R3 | 4.61 | Ohm | 0.079 | 0.548 |
| s3 | 2.82 | Ohm · s\hat{ } − ½ | 0.066 | 0.285 |

Chi²/|Z|² = 0.130;
Chi/√N = 0.332 Ω

TABLE 21
EIS fitting data for Benzonitrile 100 cycles

| | | | |
|---|---|---|---|
| Parameter | Value | Std. Error | Rank |

| | | | | |
|---|---|---|---|---|
| R1 | 7.58 | Ohm | 0.10 | 1 |
| C2 | 5.468e−05 | F | 4.18e−06 | 0.440 |
| R2 | 3.20 | Ohm | 0.0919 | 0.727 |
| C3 | 0.00195 | F | 4.53e−05 | 0.509 |
| R3 | 6.40 | Ohm | 0.052 | 0.546 |
| s3 | 2.85 | Ohm · s\hat{ } − ½ | 0.0535 | 0.278 |

Chi²/|Z|² = 0.124;
Chi/√N = 0.334 Ω

TABLE 22
EIS fitting data for Benzonitrile 150 cycles

| | | | |
|---|---|---|---|
| Parameter | Value | Std. Error | Rank |

| | | | | |
|---|---|---|---|---|
| R1 | 8.20 | Ohm | 0.1341 | 1 |
| C2 | 3.99e−05 | F | 3.46e−06 | 0.458 |
| R2 | 3.48 | Ohm | 0.124 | 0.731 |
| C3 | 0.0021 | F | 4.08e−05 | 0.571 |
| R3 | 9.95 | Ohm | 0.0769 | 0.600 |
| s3 | 3.79 | Ohm · s\hat{ } − ½ | 0.114 | 0.286 |

−Chi²/|Z|² = 0.150;
Chi/√N = 0.426 Ω

TABLE 23
EIS fitting data for Benzonitrile 200 cycles

| | | | |
|---|---|---|---|
| Parameter | Value | Std. Error | Rank |

| | | | | |
|---|---|---|---|---|
| R1 | 8.60 | Ohm | 0.148 | 1 |
| C2 | 3.50e−05 | F | 3.03e−06 | 0.468 |
| R2 | 3.88 | Ohm | 0.137 | 0.724 |
| C3 | 0.00215 | F | 3.52e−05 | 0.600 |
| R3 | 14.47 | Ohm | 0.113 | 0.596 |
| s3 | 4.35 | Ohm · s\hat{ } − ½ | 0.179 | 0.279 |

Chi²/|Z|² = 0.173;
Chi/√N = 0.518 Ω

TABLE 24
EIS fitting data for Benzonitrile 250 cycles

| | | | |
|---|---|---|---|
| Parameter | Value | Std. Error | Rank |

| | | | | |
|---|---|---|---|---|
| R1 | 9.07 | Ohm | 0.1756 | 1 |
| C2 | 3.26e−05 | F | 3.228e−06 | 0.476 |
| R2 | 3.98 | Ohm | 0.1622 | 0.725 |
| C3 | 0.00214 | F | 3.223e−05 | 0.642 |
| R3 | 19.49 | Ohm | 0.161 | 0.596 |
| s3 | 4.80 | Ohm · s\hat{ } − ½ | 0.254 | 0.276 |

Chi²/|Z|² = 0.199;
Chi/√N = 0.616 Ω

TABLE 25
EIS fitting data for Benzonitrile 300 cycles

| | | | |
|---|---|---|---|
| Parameter | Value | Std. Error | Rank |

| | | | | |
|---|---|---|---|---|
| R1 | 8.49 | Ohm | 0.179 | 1 |
| C2 | 3.135e−05 | F | 2.817e−06 | 0.492 |
| R2 | 4.575 | Ohm | 0.169 | 0.736 |
| C3 | 0.00209 | F | 3.22e−05 | 0.688 |
| R3 | 25.38 | Ohm | 0.242 | 0.602 |
| s3 | 5.519 | Ohm · s\hat{ } − ½ | 0.374 | 0.276 |

Chi²/|Z|² = 0.280;
Chi/√N = 0.769 Ω

TABLE 26
EIS fitting data for 1,2,4,5-Benzenetetracarbonitrile 50 cycles

| | | | |
|---|---|---|---|
| Parameter | Value | Std. Error | Rank |

| | | | | |
|---|---|---|---|---|
| R1 | 3.60 | Ohm | 0.047 | 1 |
| C2 | 6.88e−05 | F | 3.81e−06 | 0.417 |
| R2 | 2.02 | Ohm | 0.0452 | 0.713 |
| C3 | 0.0019 | F | 5.25e−05 | 0.491 |
| R3 | 3.41 | Ohm | 0.0433 | 0.563 |
| s3 | 3.40 | Ohm · s\hat{ } − ½ | 0.106 | 0.333 |

Chi²/|Z|² = 0.117;
Chi/√N = 0.2323 Ω

TABLE 27
EIS fitting data for 1,2,4,5-Benzenetetracarbonitrile 100 cycles

| | | | |
|---|---|---|---|
| Parameter | Value | Std. Error | Rank |

| | | | | |
|---|---|---|---|---|
| R1 | 3.72 | Ohm | 0.036 | 1 |
| C2 | 0.0001059 | F | 6.78e−06 | 0.440 |
| R2 | 1.325 | Ohm | 0.036 | 0.689 |
| C3 | 0.00217 | F | 3.69e−05 | 0.576 |
| R3 | 4.62 | Ohm | 0.044 | 0.648 |
| s3 | 4.036 | Ohm · s\hat{ } − ½ | 0.115 | 0.363 |

Chi²/|Z|² = 0.0846;
Chi/√N = 0.222 Ω

TABLE 28
EIS fitting data for 1,2,4,5-Benzenetetracarbonitrile 150 cycles

| | | | |
|---|---|---|---|
| Parameter | Value | Std. Error | Rank |

| | | | | |
|---|---|---|---|---|
| R1 | 3.920 | Ohm | 0.0444 | 1 |
| C2 | 9.52e−05 | F | 7.196e−06 | 0.446 |
| R2 | 1.370 | Ohm | 0.042 | 0.684 |
| C3 | 0.0022 | F | 3.72e−05 | 0.599 |
| R3 | 6.11 | Ohm | 0.048 | 0.654 |
| s3 | 3.96 | Ohm · s\hat{ } − ½ | 0.119 | 0.348 |

Chi²/|Z|² = 0.096;
Chi/√N = 0.227 Ω

TABLE 29
EIS fitting data for 1,2,4,5-Benzenetetracarbonitrile 200 cycles

| | | | |
|---|---|---|---|
| Parameter | Value | Std. Error | Rank |

| | | | | |
|---|---|---|---|---|
| R1 | 4.27 | Ohm | 0.051 | 1 |
| C2 | 6.398e−05 | F | 3.996e−06 | 0.454 |
| R2 | 2.01 | Ohm | 0.0502 | 0.709 |
| C3 | 0.00228 | F | 3.47e−05 | 0.596 |
| R3 | 7.894 | Ohm | 0.0681 | 0.639 |
| s3 | 4.040 | Ohm · s\hat{ } − ½ | 0.125 | 0.324 |

Chi²/|Z|² = 0.121;
Chi/√N = 0.282 Ω

TABLE 30
EIS fitting data for 1,2,4,5-Benzenetetracarbonitrile 250 cycles

| | | | |
|---|---|---|---|
| Parameter | Value | Std. Error | Rank |

| | | | | |
|---|---|---|---|---|
| R1 | 4.562 | Ohm | 0.0644 | 1 |
| C2 | 5.429e−05 | F | 3.426e−06 | 0.476 |
| R2 | 2.40 | Ohm | 0.0594 | 0.724 |
| C3 | 0.0023 | F | 3.51e−05 | 0.645 |
| R3 | 10.81 | Ohm | 0.0902 | 0.639 |
| s3 | 4.20 | Ohm · s\hat{ } − ½ | 0.152 | 0.317 |

Chi²/|Z|² = 0.142;
Chi/√N = 0.339 Ω

TABLE 31
EIS fitting data for 1,2,4,5-Benzenetetracarbonitrile 300 cycles

| | | | |
|---|---|---|---|
| Parameter | Value | Std. Error | Rank |

| | | | | |
|---|---|---|---|---|
| R1 | 4.86 | Ohm | 0.0655 | 1 |
| C2 | 5.10e−05 | F | 3.12e−06 | 0.486 |
| R2 | 2.602 | Ohm | 0.065 | 0.729 |
| C3 | 0.00232 | F | 3.146e−05 | 0.684 |
| R3 | 14.307 | Ohm | 0.119 | 0.637 |
| s3 | 4.479 | Ohm · s\hat{ } − ½ | 0.199 | 0.31 |

Chi²/|Z|² = 0.154;
Chi/√N = 0.393 Ω

TABLE 32
EIS fitting data for 3,4-Dimethoxybenzonitrile 50 cycles

| | | | |
|---|---|---|---|
| Parameter | Value | Std. Error | Rank |

| | | | | |
|---|---|---|---|---|
| R1 | 4.29 | Ohm | 0.0527 | 1 |
| C2 | 6.43e−05 | F | 3.27e−06 | 0.425 |
| R2 | 2.697 | Ohm | 0.055 | 0.728 |
| C3 | 0.00165 | F | 6.03e−05 | 0.48 |
| R3 | 3.27 | Ohm | 0.0445 | 0.560 |
| s3 | 2.623 | Ohm · s\hat{ } − ½ | 0.067 | 0.303 |

Chi²/|Z|² = 0.115;
Chi/√N = 0.221 Ω

TABLE 33
EIS fitting data for 3,4-Dimethoxybenzonitrile 100 cycles

| | | | |
|---|---|---|---|
| Parameter | Value | Std. Error | Rank |

| | | | | | |
|---|---|---|---|---|---|
| R1 | 4.5421 | Ω | 0.0501 | Ω | 1 |
| C2 | 74.21 | μF | 4.17 | μF | 0.427 |
| R2 | 2.1897 | Ω | 0.0492 | Ω | 0.699 |
| C3 | 2.0079 | mF | 50.54 | μF | 0.504 |
| R3 | 4.48 | Ω | 0.0555 | Ω | 0.559 |
| s3 | 3.6241 | Ω · s^{-1/2} | 0.1283 | Ω · s^{-1/2} | 0.312 |

Chi²/|Z|² = 0.1184;
Chi/√N = 0.2836 Ω

TABLE 34
EIS fitting data for 3,4-Dimethoxybenzonitrile 150 cycles

| | | | |
|---|---|---|---|
| Parameter | Value | Std. Error | Rank |

| | | | | |
|---|---|---|---|---|
| R1 | 4.66 | Ohm | 0.0523 | 1 |
| C2 | 8.70e−05 | F | 6.191e−06 | 0.447 |
| R2 | 1.75 | Ohm | 0.0487 | 0.695 |
| C3 | 0.00216 | F | 4.231e−05 | 0.569 |
| R3 | 5.66 | Ohm | 0.0686 | 0.634 |
| s3 | 4.154 | Ohm · s\hat{ } − ½ | 0.160 | 0.335 |

Chi²/|Z|² = 0.1107318921;
Chi/√N = 0.2995503983 Ω

TABLE 35
EIS fitting data for 3,4-Dimethoxybenzonitrile 200 cycles

| | | | |
|---|---|---|---|
| Parameter | Value | Std. Error | Rank |

| | | | | |
|---|---|---|---|---|
| R1 | 4.78 | Ohm | 0.054 | 1 |
| C2 | 8.94e−05 | F | 6.90e−06 | 0.447 |
| R2 | 1.56 | Ohm | 0.052 | 0.683 |
| C3 | 0.00224 | F | 3.58e−05 | 0.597 |
| R3 | 6.95 | Ohm | 0.072 | 0.640 |
| s3 | 4.265 | Ohm · s\hat{ } − ½ | 0.161 | 0.338 |

Chi²/|Z|² = 0.105;
Chi/√N = 0.296 Ω

TABLE 36
EIS fitting data for 3,4-Dimethoxybenzonitrile 250 cycles

| | | | |
|---|---|---|---|
| Parameter | Value | Std. Error | Rank |

| | | | | |
|---|---|---|---|---|
| R1 | 4.88 | Ohm | 0.0585 | 1 |
| C2 | 8.67e−05 | F | 7.99e−06 | 0.445 |
| R2 | 1.44 | Ohm | 0.0546 | 0.677 |
| C3 | 0.00230 | F | 3.44e−05 | 0.617 |
| R3 | 8.37 | Ohm | 0.074 | 0.640 |
| s3 | 4.302 | Ohm · s\hat{ } − ½ | 0.160 | 0.333 |

Chi²/|Z|² = 0.103;
Chi/√N = 0.296 Ω

TABLE 37
EIS fitting data for 3,4-Dimethoxybenzonitrile 300 cycles

| | | | |
|---|---|---|---|
| Parameter | Value | Std. Error | Rank |

| | | | | | |
|---|---|---|---|---|---|
| R1 | 5.080 | Ω | 0.0595 | Ω | 1 |
| C2 | 70.07 | μF | 6.18 | μF | 0.447 |
| R2 | 1.62 | Ω | 0.057 | Ω | 0.681 |
| C3 | 2.28 | mF | 32.87 | μF | 0.621 |
| R3 | 9.904 | Ω | 0.0850 | Ω | 0.632 |
| s3 | 4.25 | Ω · s^{-1/2} | 0.162 | Ω · s^{-1/2} | 0.323 |

Chi²/|Z|² = 0.113;
Chi/√N = 0.320 Ω

### Analysis and Characterization

To investigate the origin of the effect of 3,4-dimethoxybenzonitrile, two batteries identical to those previously cycled-one reference and one containing 3,4-dimethoxybenzonitrile-were stopped after 50 cycles and subsequently analyzed using XPS and TEM.

To better evaluate the impact of the additive on the improved cyclability, an XPS analysis was performed to determine whether 3,4-dimethoxybenzonitrile or any cycling-induced derivatives were present on the cathode surface. A survey spectrum was acquired for both the reference electrode and the electrode containing the additive. In both survey spectra (FIG. 5), the major detected elements were C, O, F, and Li. A weak Ni 2p signal originating from the underlying NMC particles was also observed, whereas no Co or Mn signals were detected. This suggests that a carbonaceous surface layer formed on the NMC particles, thick enough that most photoelectrons generated within the bulk NMC are reabsorbed before escaping.

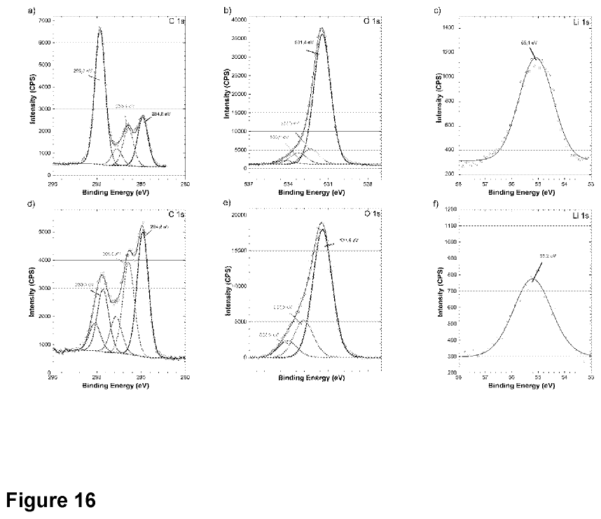

High-resolution spectra were acquired for C 1s, O 1s, Li 1s, F 1s, and N 1s to characterize the chemical species present. In the C 1s spectra of both samples (FIG. 16a,c), peaks at 284.8 eV and 286.4 eV were observed and assigned to C—C/C—H (sp³) and C*—C—F environments, respectively. These features arise from the carbon black and PVDF components of the composite electrode. A peak at 289.3 eV was also detected and attributed to CO₃²⁻ species. The presence of carbonate is further confirmed by the O 1s spectra (FIG. 16b,e), which exhibit a dominant peak at 531.4 eV characteristic of carbonate environments, yielding the expected C: O atomic ratio of 1:3. Overall, these results confirm the formation of carbonate species on the surface e of both electrodes, with or without the 3,4-dimethoxybenzonitrile additive, consistent with electrolyte decomposition products. In addition, a peak at 55.2 eV in the Li 1s spectra (FIG. 16c,f), observed in both samples, along with an elemental ratio Li:C:O of 2:1:3, is consistent with the presence of Li₂CO₃.

A key difference between electrodes cycled with and without the additive lies in the relative intensity of the CO₃²⁻ peak. Its lower intensity in the presence of the additive suggests a reduced extent of electrolyte decomposition.

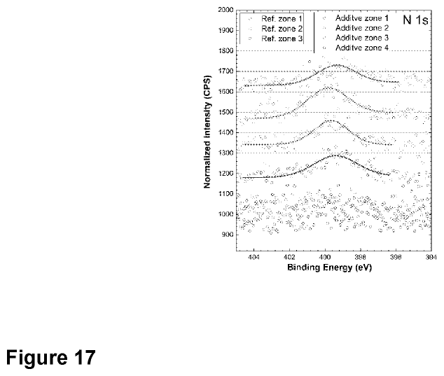

Nitrogen from the nitrile group in the additive is the only element absent from the reference sample; however, its concentration in the cycled electrode remains very low. To maximize the likelihood of detecting the nitrile functionality, the N 1s spectrum was acquired over an extended acquisition time compared with the other core levels. In addition, to minimize the risk of measurement artifacts, XPS analyses were performed at four different locations on the samples containing the additive and at three locations on the reference electrode. The results are presented in FIG. 17. For clearer comparison, all spectra were normalized to the same intensity scale. A nitrogen peak is observed at approximately 399 eV in the cycled sample containing the additive. For most neutral organic nitrogen environments, the N 1s binding energy typically lies between 399 and 400 eV, consistent with the presence of a nitrile group. However, due to the low signal-to-noise ratio, an unambiguous peak assignment remains challenging.

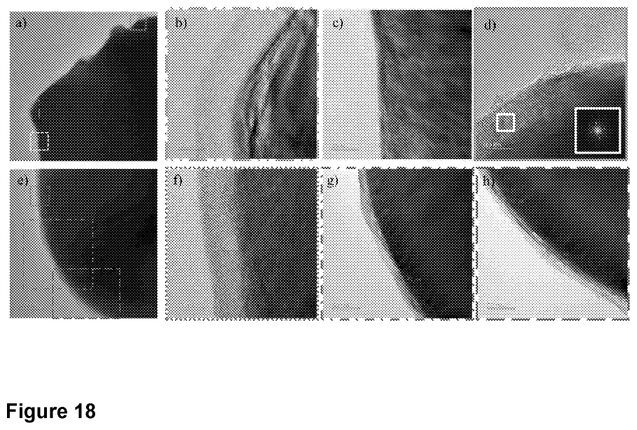

To further clarify the origin of the enhanced cycling performance observed when 3,4-dimethoxybenzonitrile is added to the electrolyte, BF-TEM images were acquired on the NMC particles (FIG. 18a-h). The sample cycled with the additive exhibits a continuous surface layer approximately 10-30 nm thick (FIG. 18e-h), whereas the sample without the additive shows a heterogeneous interphase, ranging from the absence of a CEI to regions where a layer up to ~15 nm is present (FIG. 18a-d). Such morphological differences suggest a more controlled and uniform interfacial evolution in the presence of the additive. It is widely accepted that achieving a stable and homogeneous CEI is critical to preventing parasitic interfacial reactions and ensuring long-term electrode integrity [30,31].

These embodiments of the present invention highlight the effectiveness of 3,4-dimethoxybenzonitrile as a multifunctional electrolyte additive for improving the cyclability of lithium-ion batteries using NMC 811 as the active material. By systematically comparing additives with distinct functional groups—nitrile, methoxy, and benzyl—the synergy between the nitrile and dimethoxy groups in 3,4-Dimethoxybenzonitrile were demonstrated. This synergy plays a critical role in stabilizing the cathode-electrolyte interphase (CEI). Among all tested additives, 3,4-Dimethoxybenzonitrile exhibited the most significant improvement in long-term cycling performance, achieving nearly double the cycle life at 90% capacity retention compared to the reference electrolyte.

Electrochemical impedance spectroscopy (EIS) and coulombic efficiency analyses further confirmed the superior performance of this additive. Notably, the charge transfer resistance (Rct) remained lower than the reference throughout extended cycling, indicating the formation of a stable and non-resistive CEI. Subsequent analyses, including XPS, revealed the presence of nitrogen-containing species on the cathode surface, supporting the hypothesis that the nitrile group contributes to CEI formation. Moreover, TEM analysis confirms the formation of a uniform CEI layer with an average thickness of approximately 10 to 30 nm.

These findings underscore the importance of rational additive design based on functional group synergy. Multifunctional additives can simultaneously enhance interfacial stability and minimize irreversible capacity loss.

The additive for use in association with the electrolyte is adapted to be compatible with the components of the battery including the electrolyte and the cathode active material.

Aspects of the invention are described in relation to lithium manganese iron phosphate (LMFP)-lithium titanium oxide (LTO) batteries. As will be understood by a skilled person, other lithium-ion battery types may also be used. In other words, any battery wherein the cathode active material comprises a lithium-containing material may be used. Such lithium-containing material may be lithium cobalt oxide (LCO), lithium manganese oxide (LMO), lithium nickel oxide (LNO) and the like, including olivines, lithium oxides, nickel manganese cobalt oxide (NMC).

The anode material can be of any suitable type, such as for example lithium alloys, Si, SiOx, graphite and carbon mixtures, titanates, lithium titanates.

The scope of the claims should not be limited by the disclosed embodiments set forth in the examples but should be given the broadest interpretation consistent with the description as a whole.

1. A method of improving the performance and safety of a Li-ion battery, comprising using a nitrile-based organic compound in association with the electrolyte of the battery, wherein the compound has a general formula A0 outlined below 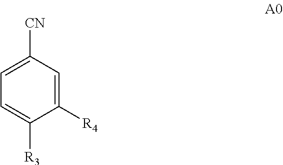
wherein R₃ and R₄ are each independently a C1-C6 alkoxy or a thioalkoxy.

2. The method according to claim 1, wherein R₃ and R₄ are each independently a C1-C6 alkoxy.

3. The method according to claim 1, wherein R₃ and R₄ are the same.

4. The method according to claim 1, wherein at least one of R₃ and R₄ is OCH₃.

5. The method according to claim 1, wherein both R₃ and R₄ are OCH₃ and the compound is 3,4-dimethoxybenzonitrile outlined below

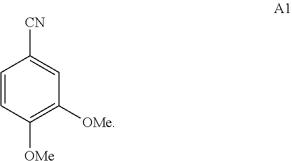 [SMILES: COc1ccc(C#N)cc1OC]

6. The method according to claim 1, wherein the nitrile-based organic compound is added to the electrolyte; optionally an amount of the additive (nitrile-based organic compound) is between about 0.01 to about 5.0% wt, or about 0.01 to about 3.0% wt, or about 0.01 to about 1.0% wt, or about 0.05 to about 1.0% wt, or about 0.1 to about 1.0% wt, about 0.1 to about 0.8% wt, or about 0.1 to about 0.5% wt, or about 0.1 to about 0.3% wt, is 0.1% wt, or is 0.5% wt.

7. The method according to claim 1, wherein the nitrile-based organic compound is added to the electrolyte in an amount ranging between about 0.01 to about 5.0% wt.

8. The method according to claim 1, wherein the nitrile-based organic compound is added to the electrolyte in an amount ranging between about 0.1 to about 0.5% wt.

9. An additive for an electrolyte for use in a Li-ion battery, comprising a nitrile-based organic compound having a general formula AO outlined below 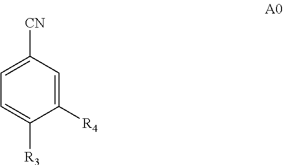
wherein R₃ and R₄ are each independently a C1-C6 alkoxy or a thioalkoxy.

10. The additive according to claim 9, wherein the compound is 3,4-dimethoxybenzonitrile.

11. An electrolyte for a Li-ion battery, comprising a nitrile-based organic compound having a general formula A0 outlined below 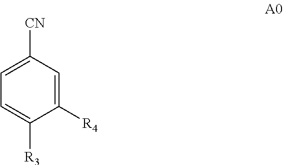
wherein R₃ and R₄ are each independently a C1-C6 alkoxy or a thioalkoxy.

12. The electrolyte according to claim 11, wherein the compound is 3,4-dimethoxybenzonitrile.

13. A battery comprising the electrolyte as defined in claim 11.

14. The method according to claim 1, wherein a cathode of the Li-ion battery comprises a lithium-containing material.

15. The method according to claim 1, wherein a cathode of the Li-ion battery comprises lithium cobalt oxide (LCO), lithium manganese oxide (LMO), lithium nickel oxide (LNO) and the like including olivines, lithium oxides, nickel manganese cobalt oxide (NMC).

16. The method according to claim 1, wherein the performance (capacity, reversibility) of the battery is improved.

17. The method according to claim 1, wherein the nitrile group and the C1-C6 alkoxy or thioalkoxy act synergistically on an overall improvement of the performance (capacity, reversibility) of the battery.

18. The method according to claim 1, wherein the nitrile group and the C1-C6 alkoxy or thioalkoxy act synergistically in stabilizing the cathode-electrolyte interphase (CEI).

19. The method according to claim 1, wherein a stable and non-resistive cathode-electrolyte interphase (CEI) is formed.

20. The method according to claim 1, wherein a uniform cathode-electrolyte interphase (CEI) layer is formed, which has an average thickness of about 10 to about 30 nm is formed.
